(19) United States
(12) Patent Application Publication
(10) Pub. No.: US 2024/0050743 A1
(43) Pub. Date: Feb. 15, 2024

(54) APPARATUS AND METHOD FOR DIAPHRAGM STIMULATION

(71) Applicant: Stimdia Medical, Inc., Mendota Heights, MN (US)

(72) Inventors: Ishmael Bentley, II, Edina, MN (US); Stephen Lorne Bolea, Excelsior, MN (US); Frank Francis Jocewicz, III, Minneapolis, MN (US); Tim Miller, Inver Grove Heights, MN (US)

(21) Appl. No.: 18/229,305

(22) Filed: Aug. 2, 2023

Related U.S. Application Data
(60) Provisional application No. 63/441,300, filed on Jan. 26, 2023.
(60) Provisional application No. 63/397,143, filed on Aug. 11, 2022.

(51) Int. Cl. A61N 1/36; A61M 16/00; A61N 1/05; A61N 1/372

(52) U.S. Cl. CPC A61N 1/3601; A61M 16/0003; A61N 1/0553; A61N 1/36139; A61N 1/37247; A61N 1/0502; A61N 1/37258; A61M 2205/054

(57) ABSTRACT

A number of systems and methods described here are directed to controlling, activating, and otherwise interacting with the phrenic nerve of a patient while the patient is undergoing mechanical ventilation, thus interacting with the diaphragm. Some embodiments of the systems and methods described herein are configured to achieve phrenic nerve stimulation in a controlled manner so as to sufficiently adapt the breathing therapy to a variety of patients.

### CROSS-REFERENCE TO RELATED APPLICATIONS

This application claim priority to and benefit of U.S. Provisional Application No. 63/441,300, filed on Jan. 26, 2023, and 63/397,143, filed on Aug. 11, 2022, the contents of which are incorporated by reference in their entirety.

### TECHNICAL FIELD

This disclosure relates to medical devices, systems and their methods of use in breathing, such as those that may involve mechanical ventilation. Some implementations described herein are directed to providing electrical stimulation of the phrenic nerve of a patient undergoing augmented breathing, such as via mechanical ventilation, assistive breathing approaches in which a patient may initiate a breath, continuous positive airway pressure (CPAP) use, and applications involving sleep apnea. Such approaches may facilitate ventilation and patient recovery, for instance by mitigating effects of ventilator-induced diaphragmatic dysfunction.

### BACKGROUND

Augmented breathing approaches such as mechanical ventilation may be used clinically to maintain gas exchange in patients to assist in maintaining adequate alveolar ventilation. It can be desirable to avoid prolonged mechanical ventilation, and to assist in weaning patients off assisted breathing. For example, even though mechanical ventilation can be a life-saving intervention for patients suffering from respiratory failure, prolonged mechanical ventilation can promote diaphragmatic atrophy and contractile dysfunction, which is referred to as ventilator-induced diaphragm dysfunction. In some circumstances, mechanical ventilation of a patient for a period of only a few days can be sufficient to develop ventilator-induced diaphragm dysfunction.

Stimulating the phrenic nerve is a manner in which to facilitate augmented breathing, and it can be used to maintain some level of diaphragm activity of a patient on a mechanical ventilator. When the patient is on a ventilator and unable to self-generate inspiratory effort, phrenic nerve pacing can be used to induce work in the diaphragm muscle and thereby reduce or reverse atrophy, especially in severe cases where the patient has become ventilator dependent and requires a training regime of pacing to strengthen their muscles.

### SUMMARY

Some embodiments of the systems and methods described herein are configured to achieve phrenic nerve stimulation in a controlled manner so as to sufficiently adapt the breathing therapy to a variety of patients, including patients that experience a change in levels of patient respiratory effort over time. In particular implementations, the system can effect such control in an efficient (computationally and otherwise) and rapid manner such that dynamic response can be achieved. Some examples detailed herein provide a system for diaphragm stimulation that is specifically programmed to monitor one or more sensor signals in real time during an inspiratory phase of a patient and to controllably adapt a stimulation intensity level in a manner that avoids harsh changes to stimulation intensity during a next stimulated breath and that accounts for momentary outlier values from the sensor signals.

Particular embodiments described herein include a phrenic nerve stimulation system that includes a phrenic nerve stimulation control console and a phrenic nerve stimulation lead assembly. The phrenic nerve stimulation control console can be configured to control a stimulation intensity of a phrenic nerve stimulation signal deliverable to at least one phrenic nerve during mechanical ventilation. The phrenic nerve stimulation lead assembly may include at least one electrode lead positionable proximate to the at least one phrenic nerve to output the phrenic nerve stimulation signal from the phrenic nerve stimulation control console to the corresponding phrenic nerve. In some optional implementations, in response to a detected level of patient respiratory effort being outside a predetermined range, the phrenic nerve stimulation control console can controllably increment or decrement the stimulation intensity of the phrenic nerve stimulation signal by a preset increment value.

In further embodiments, a phrenic nerve stimulation system may include a phrenic nerve stimulation control console, a phrenic nerve stimulation lead assembly, and a muscle activity sensor. The phrenic nerve stimulation control console can be configured to control a stimulation intensity of a phrenic nerve stimulation signal deliverable to at least one phrenic nerve during mechanical ventilation. The phrenic nerve stimulation lead assembly may include at least one electrode lead positionable proximate to the at least one phrenic nerve to output the phrenic nerve stimulation signal from the phrenic nerve stimulation control console to the corresponding phrenic nerve. The muscle activity sensor can be in communication with the phrenic nerve stimulation control console to provide feedback indicative of muscle activity in at least one of a shoulder and neck. In some optional implementations, in response to a detected level of muscle activity being greater a predetermined threshold, the phrenic nerve stimulation control console can decrement the stimulation intensity of the phrenic nerve stimulation signal by a preset increment value.

Additional embodiments described herein include a method of controlling phrenic nerve stimulation. The method may include, after delivering a first cycle of phrenic nerve stimulation at a first intensity level from a phrenic nerve stimulation apparatus to at least one stimulation lead proximate to at least one phrenic nerve, delivering a second cycle of phrenic nerve stimulation at the first intensity level from the phrenic nerve stimulation apparatus to the at least one stimulation lead proximate to the at least one phrenic nerve. Optionally, the method may also include, in response to detecting that a parameter indicative of diaphragm contractile force is greater during the second cycle than the first cycle, outputting an alert via a user interface of the phrenic nerve stimulation apparatus indicative of a readiness to wean from mechanical ventilation.

Some embodiments described herein include a method of phrenic nerve stimulation. The method may include, during a first stimulated breath of a patient in which at least one phrenic nerve is electrically stimulated by a phrenic nerve stimulation signal delivered from a phrenic nerve stimulation control console to at least one stimulation lead positioned proximate to the at least one phrenic nerve, delivering the phrenic nerve stimulation signal during a full stimulation period. The method may also include detecting a level of patient respiratory effort during the first stimulated breath. Further, the method may include, during a second stimulated breath of the patient, delivering a notched stimulation signal from a phrenic nerve stimulation control console to at least one stimulation lead positioned proximate to the at least one phrenic nerve so that electrical stimulation is temporarily terminated for portion of the full stimulation period. The method may include outputting an alert via a user interface of the phrenic nerve stimulation apparatus indicative of a readiness to wean from mechanical ventilation in response to detecting a particular condition. Optionally, detecting the particular condition can be either: detecting a reduction in an airflow characteristic during the notched stimulation signal is less than a threshold value, or detecting a magnitude of the reduction in the airflow characteristic during notched stimulation signal is less than or equal to previous magnitude detected during a previous delivery of the notch stimulation signal.

A number of embodiments described herein include a mechanical ventilator configured to deliver breathable air to lungs of a patient. The mechanical ventilator may include a ventilator control console configured to control a selected pressure or flow parameter of a respiration therapy breath during mechanical ventilation. Also, the mechanical ventilator may include a breathing circuit tube to direct the respiration therapy breath to the patient. In some optional implementations, the mechanical ventilator may include a phrenic nerve stimulation lead assembly connected to the ventilator control console via cable and including at least one electrode lead positionable proximate to the at least one phrenic nerve to output a phrenic nerve stimulation signal during the respiration therapy breath. The ventilator control console of the mechanical ventilator can optionally include a graphic user interface to control a stimulation intensity of the phrenic nerve stimulation signal deliverable to the at least one phrenic nerve during mechanical ventilation.

Further embodiments described herein include a stimulation electrode and stimulation circuitry to stimulate the diaphragm of a patient. The stimulation circuitry can stimulate the diaphragm by delivering, via the stimulation electrode, electrical energy to a phrenic nerve of the patient at a stimulation intensity, including maintaining a characteristic respiratory airflow parameter of the patient within a target range by incrementally increasing or decreasing the stimulation intensity by a predetermined incremental amount.

In some embodiments described herein, an apparatus may include stimulation circuitry to apply stimulation via one or more electrodes, and control circuitry. The control circuitry may be optionally configured to: stimulate the diaphragm of a patient by delivering electrical energy to a phrenic nerve of the patient via the stimulation circuitry, coincident with inspiration; record and track an amount of breathing effort of the patient for a given intensity level of electrical stimulation; and generate and output an indication signal in response to the patient achieving a target level of breathing effort.

Additionally or alternatively, some embodiments include an apparatus that includes stimulation circuitry to apply stimulation via one or more electrodes, and control circuitry. The control circuitry may be optionally configured to: assess respiratory effort of a patient for a given intensity level of electrical stimulation applied to the patient's phrenic nerve via the stimulation circuitry; and generate and output an indication in response to the assessed respiratory effort reaching a plateau at which gains in effort for a given stimulation intensity over time diminish.

Further embodiments described herein include an apparatus that includes stimulation circuitry to apply stimulation via one or more electrodes, and control circuitry. The control circuitry may be optionally configured to enhance rate at which a patient's diaphragm is strengthened or rehabilitated by stimulating the patient's diaphragm, with electrical energy delivered to a phrenic nerve of the patient via the stimulation circuitry, on successive breaths using a predetermined varying pattern of stimulation intensity levels.

In some embodiments, an apparatus that includes stimulation circuitry to apply stimulation via one or more electrodes, and control circuitry. The control circuitry may be optionally configured to stimulate a patient's diaphragm through delivery of electrical energy to a phrenic nerve of the patient coincident with inspiration, based on a representative airflow parameter indicating that the patient is experiencing hypoventilation or central sleep apnea, by providing a diaphragmatic pacing function.

Particular embodiments described herein include an apparatus that includes lead circuitry and processing circuitry. The lead circuitry can be configured to couple stimulation energy to a phrenic nerve of a patient having a diaphragm. The processing circuitry can be configured to stimulate the diaphragm by delivering stimulation energy to the phrenic nerve via the lead circuitry, including incrementally increasing or decreasing the stimulation energy by a predefined amount on subsequent breaths of the patient based on a characteristic respiratory airflow parameter of the patient and a target range for the parameter.

One or more of the embodiments described herein may be configured to achieve a number of advantages. First, some embodiments of a phrenic nerve stimulation system can configured to provide adaptive stimulation during mechanical ventilation, including a control system that is adaptive to detected levels of a patient's respiratory effort. Second, particular embodiments described herein can be configured to predictively anticipate a patient's readiness for weaning from mechanical ventilation based on, for example, sensor data input into the control system over time. Third, some embodiments of phrenic nerve stimulation system may connected to and controlled by a mechanical ventilator console. Fourth, in some implementations, a phrenic nerve stimulation system can receive user input indicative of an increment value for changes to stimulation intensity and can thereafter perform automated, incremental changes to the stimulation intensity level (e.g., an increase or decrease by the increment value) based upon a detected level of patient respiratory effort, thereby providing a safe degree of adaptive control configured to avoid large or uncomfortable fluctuations in stimulation intensity from one stimulated breath to a next stimulated breath. Fifth, some embodiments of a phrenic nerve stimulation system can efficiently and automatically detect an operating range of stimulation intensity levels (e.g., including a maximum level of stimulation intensity customized for a particular patient) based upon detect levels of patient respiratory effort during mechanical ventilation. Fifth, a number of embodiments of a phrenic nerve stimulation system can be configured to automatically decrement a stimulation intensity level in response to sensing muscle activity in at least one of a patient's neck and shoulder. Sixth, some embodiments of a phrenic nerve stimulation system can advantageously include a user interface that outputs an alert indicative of a patient's readiness to wean from mechanical ventilation in response to detecting, for example, changes to a sensed diaphragm contractile force. Seven, in some version, a phrenic nerve stimulation system can be controlled to implement a predetermined training pattern for stimulating the phrenic nerve using a sequence of differing stimulation intensity levels during a training session.

The above overview is not intended to describe each embodiment or every implementation of the present disclosure. The figures, detailed description and claims that follow also exemplify various embodiments. Other features, objects, and advantages will be apparent from the description and drawings, and from the claims.

### DESCRIPTION OF DRAWINGS

FIG. 1 is a perspective view of a system for diaphragm stimulation, in accordance with some embodiments.

FIG. 2 is a perspective view of a phrenic nerve stimulation apparatus of the system of FIG. 1.

FIG. 3A is another perspective view of the phrenic nerve stimulation apparatus of the system of FIG. 1.

FIGS. 3B, 3C, and 3D are perspective views of alterative stimulation leads of the phrenic nerve stimulation apparatus of FIG. 3A.

FIG. 4 is a perspective view of an alternative system for diaphragm stimulation, in accordance with further embodiments.

FIG. 5A is a flow chart of an example process of stimulation control, in accordance with some embodiments.

FIG. 5B is a diagram of examples of a phrenic nerve stimulation signal from the process of FIG. 5A

FIG. 6 is a flow chart of an example process of stimulation control, in accordance with some embodiments.

FIG. 7 is a flow chart of an example process of stimulation control, in accordance with some embodiments.

FIG. 8A is a flow chart of an example process of detecting a readiness to wean from mechanical ventilation, in accordance with some embodiments.

FIG. 8B is a diagram of examples of a phrenic nerve stimulation signal from the process of FIG. 8A.

FIG. 9A is a flow chart of an example process of detecting a readiness to wean from mechanical ventilation, in accordance with some embodiments.

FIG. 9B is a diagram of examples of a phrenic nerve stimulation signal from the process of FIG. 9A

FIG. 10 is a flow chart of an example process of delivering phrenic nerve stimulation in combination with mechanical ventilation, in accordance with some embodiments.

FIG. 11 is a flow chart of an example process of delivering phrenic nerve stimulation in combination with mechanical ventilation, in accordance with some embodiments.

FIG. 12A is a flow chart of an example process of stimulation control, in accordance with some embodiments.

FIGS. 12B-C are diagrams of examples of a phrenic nerve stimulation signal from the process of FIG. 12A.

FIG. 13 is an example of breath sensor signals, including airway flow and pressure signals that can be displayed on a graphic user interface of some example systems described herein, in accordance with particular embodiments.

Like reference symbols in the various drawings indicate like elements. Furthermore, the various figures and charts as shown may be utilized independently, in connection with one another, or modified based on other aspects disclosed herein.

### DETAILED DESCRIPTION

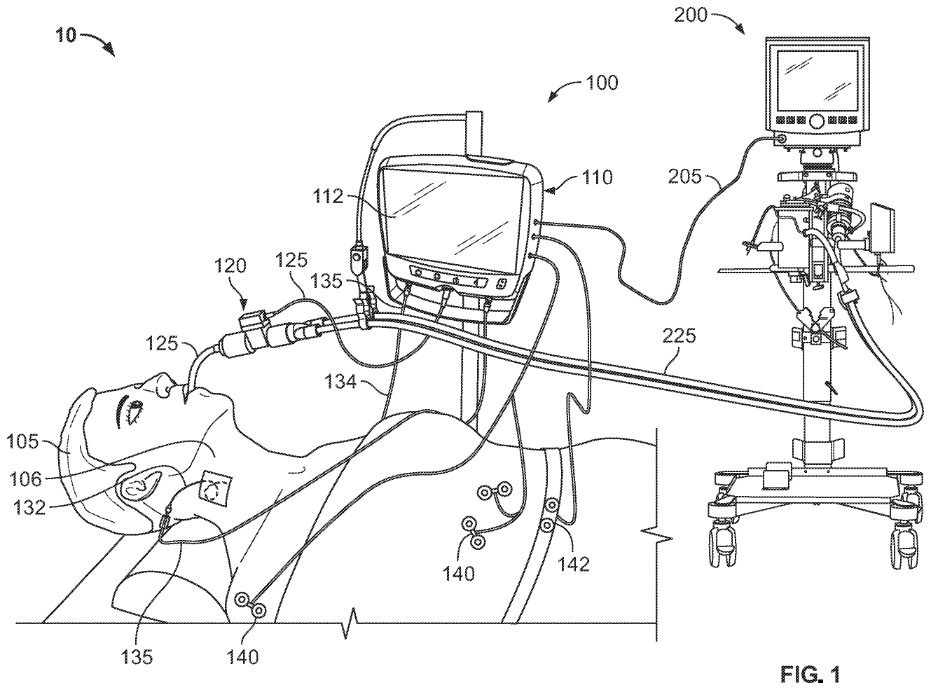

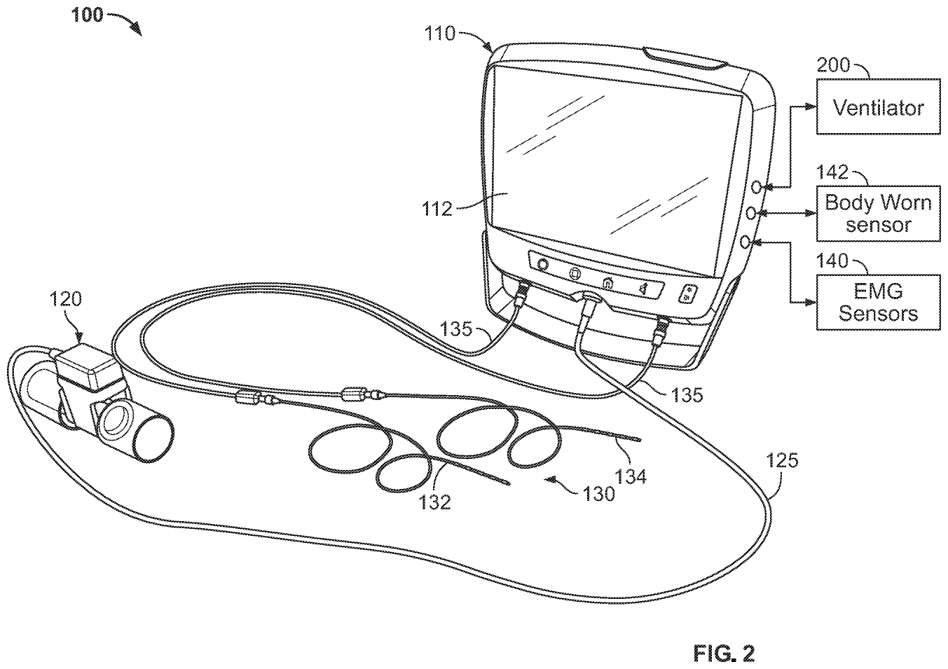

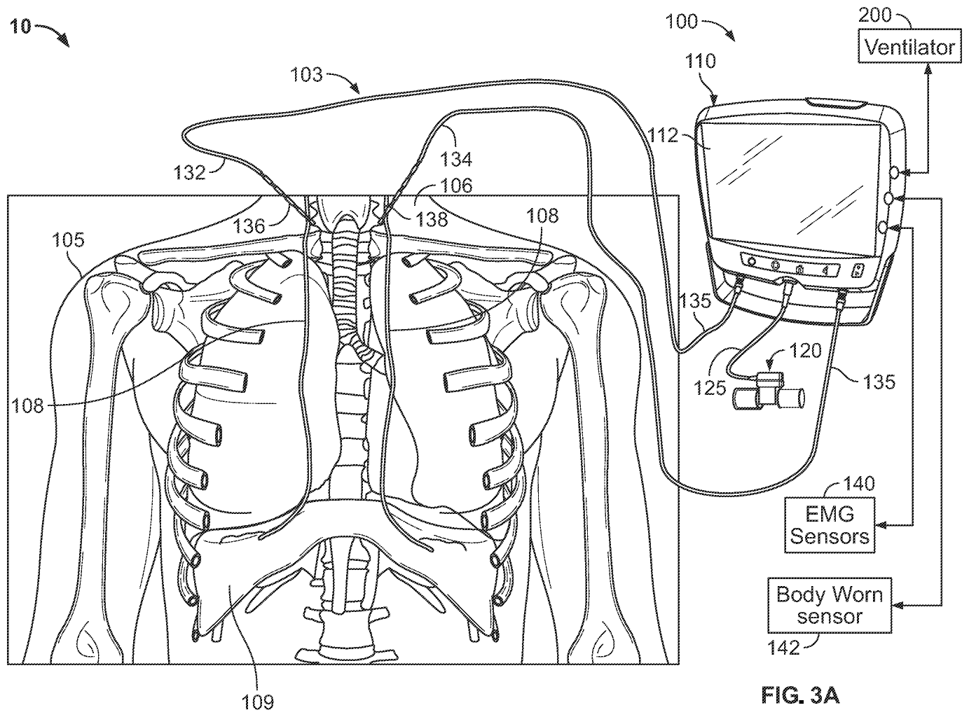

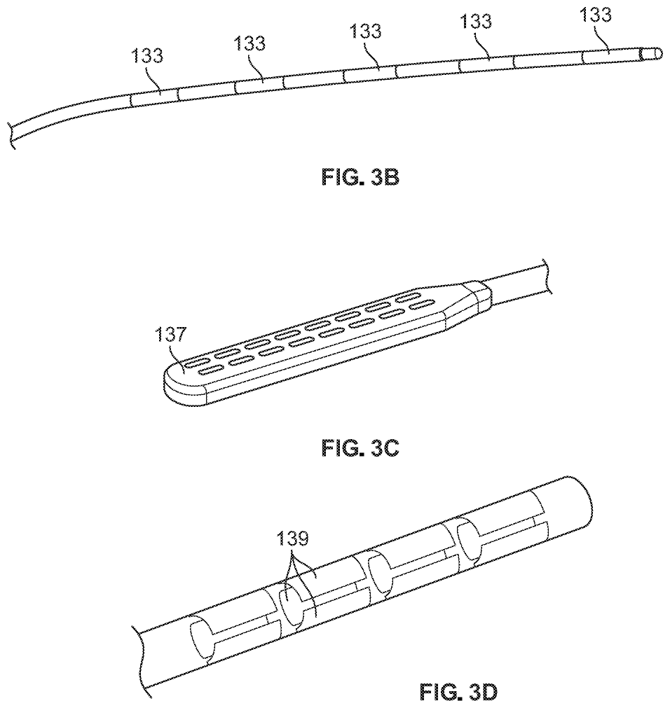

Referring now to FIGS. 1-3D, some embodiments of system 10 for diaphragm stimulation include a phrenic nerve stimulation apparatus 100 and a mechanical ventilator apparatus 200. The phrenic nerve stimulation apparatus 100 can include a control console 110 in communication with a breath sensor 120 and an electrical stimulation lead assembly 130. The breath sensor 120 can be in the form of a wye sensor, which is coupled to an air flow path extending between an intubation conduit 125 positioned in an airway of a patient 105 and a breathing circuit tubing 225 of the mechanical ventilator 200. The lead assembly 130 can include one or more leads configured to output electrical stimulation to corresponding phrenic nerves. In the depicted embodiment, the lead assembly 130 includes two percutaneous leads 132 and 134 (e.g., a right lead and a left lead; refer also to FIGS. 2-3D), which are configured for percutaneous insertion into a neck 106 of a patient 105 such that a selected electrode pair of the right lead 132 captures a right phrenic nerve and another selected electrode pair of the left lead 134 captures a left phrenic nerve.

As described in more detail below, the control console 110 includes a graphic user interface 112 (e.g., a touchscreen and, optionally, mechanical switches for user input), a computer-readable memory and central processing unit positioned within the housing of the console 110 and configured to control stimulation of the patient's diaphragm according to the implementations detailed below, multiple connection ports to receive sensor data (e.g., from the wye sensor 120, from a number of optional EMG sensors 140, from an optional force sensor 142 worn on the patient's body, and others), a connection port for data communication with the mechanical ventilator 200, and one or more ports for connections with corresponding cables 135 the lead assembly 130. The control console 110 is programmed to, responsive to one or more sensed conditions, deliver electrical current to the lead assembly 130 for purposes of electrically stimulating one or both of the phrenic nerves 108 (FIG. 3A), which thereby activate the diaphragm 109 of the patient 105 while the patient is undergoing mechanical ventilation (from the ventilator 200). The wye sensor 120 can optionally include at least one flow sensor configured to detect a number of breathing characteristics (e.g., pressure, flow direction, volume, and the like) that are input to the control console 110 and used to monitor the patient and determine whether a sufficient level of stimulation is applied to the patient's diaphragm 109. For example, wye sensor 120 can pneumatically connected to the breathing circuit tubing 225 of the mechanical ventilator 200 to measure both flow and pressure using standard differential and gauge pressure sensors within the desired range of operation. In this embodiment, the wye sensor 120 is electrically coupled to the control console 110 via a cable 125. As previously described, the control console 110 houses a processor and computer-readable memory, which controls an integrated pulse generator to supply an electrical output delivered to the leads 132 and 134 via the corresponding lead cables 135. In some embodiments, data received from the wye sensor 120, lead assembly 130, the optional EMG sensors 140 and body worn sensor 142, and the ventilator 200, as well as the output parameters of the pulse generator, can be displayed on the touchscreen display of the control console 110. The computer-readable memory device housed within the control console 110 stores software that performs the stimulation control tasks, including for example reading input data from the above-described sensors, performing algorithmic calculations, and setting outputs based upon the user set inputs and algorithmic calculations. In some optional implementations, the control console 110 can be configured to export patient data, including sensor data and historical phrenic nerve stimulation data, to an external memory device (e.g., a USB memory device or external hard drive) or an external computing device (e.g., via a wireless network connection, a Bluetooth connection, or a cable connection). For example, particular implementations, the patient data can be exported for purposes of real-time data processing at the external computing device, which is configured for a greater computation load or other predictive modeling that can assess when a patient is ready to wean from mechanical ventilation, so that the external computing device can communicate back to the control console 110 for outputting an alert or other information on the graphic user interface 112.

Referring to FIGS. 3A-D, the lead assembly 130 can include one or more stimulation leads, and in the depicted example of FIG. 3A, each of the right and left leads 132 and 134 includes a unitary lead body having a distal end with multiple electrodes and a proximal end having a set of four terminals for connection to the control console 110 (via the corresponding cable 135). In this embodiment, each lead 132 and 134 is a multipolar lead having at least four electrodes (axially spaced apart) exposed along a percutaneous insertion tip 136, 138 of the lead 132, 134, respectively. Alternatively, each lead 132 and 134 shown in FIG. 3A can instead have five ring electrodes 133 axially spaced apart from another (as depicted, for example, in FIG. 3B). In another alternative, each lead 132 and 134 shown in FIG. 3A can include a percutaneously insertable paddle electrode lead tip 137 (as depicted, for example, in FIG. 3C) having an array of electrodes (e.g., a 2×8 array of sixteen electrodes in this example) along a planar surface. Alternatively, each lead 132 and 134 shown in FIG. 3A can include multiple segmented electrodes 139 (as depicted, for example, in FIG. 3C) spaced apart along a cylindrical portion of the lead tip along with additional sets of segmented electrodes that are axially spaced apart from one another (e.g., provide a set of twelve total electrodes in the depicted example. In some implementations, the different types of the electrode leads 132 and 134 (refer to the examples in FIGS. 3A, 3B, 3C, and 3D) can be provided to a user, and the user may select a particular type of electrode lead depending upon the lead insertion orientation of the percutaneous insertion tip for each lead 132 and 134 and the particular anatomy of in the patient's neck 106.

Still referring to FIGS. 3A-D, at least one pair of electrodes along each lead 132 and 134 can be anchored proximate to the phrenic nerve 108 such that a selected electrode pair may be positioned traverse to the phrenic nerve 108 in the neck 106 and electrically capture the nerve 108 (in response to delivery of an electrical stimulation signal output from the selected electrode pair) for purposes of stimulating the diaphragm 109. For example, the control sole can be configured to initiate an electrode selection process that activates a number of electrode pair options and detects a level of stimulation (e.g., via a detected work of breathing, via the EMG sensors, or a combination thereof) for each of the electrode pair options, in which case the control console can automatically select one of the electrode pair options that sufficiently captures the phrenic nerve (to activate the diaphragm) with a lower electrical current input level. Alternatively, the control console may provide a recommendation of the one of the electrode pair options, and the user may manually select the recommended electrode pair using the touchscreen interface 112. Additionally or alternatively, the user may manually initiate the stimulation of electrode pair options, and then manually select (using the touchscreen interface 112) one of the electrode pair options that sufficiently captures the phrenic nerve (to activate the diaphragm) with a lower electrical current input level. The control console 110 can be implemented to provide both unipolar and bipolar stimulation options using the lead assembly, with both anodal and cathode stimulation available for the therapeutic use. Also, the control console 110 can be implemented to provide both monophasic and balanced and unbalanced biphasic current stimulation, and in the example depicted in FIG. 3A, the control console 110 is configured to deliver biphasic stimulation from a selected electrode pair (on each of the leads 132 and 134) to achieve a satisfactory level of work of breathing (as calculated by the control console 110 described below). Additionally, the selected electrode pair can be modified (e.g., to select a different pair) after a period of time or in response to detecting the lead 132, 134 has migrated relative to the nerve 108 over time.

In use, the control console 110 of FIGS. 1-3A can be configured to deliver phrenic nerve stimulation during a subset of breaths while the patient is subject to mechanical ventilation from the ventilator 200. The human breath has an inspiratory phase characterized by a positive flow of air through the wye sensor 120 into the patient, and an exhalation phase which begins when flow through the wye sensor reverses (e.g., the flow value drops below zero and turns negative) as the patient exhales the volume just inspired. This end of inspiration event typically begins the outflow portion of the breath cycle. In operation, the control console 110 is programmed to deliver the electrical stimulation starting with the inspiratory phase (of a designated stimulated breath) when flow exceeds a first predetermined level and end stimulation no later than the start of the exhalation phase when flow drops below a second predetermined level. Not all breaths of the patient during mechanical ventilation need be stimulated breaths. In many embodiments, the control console 110 will deliver phrenic nerve stimulation only during the designated stimulation breath between a predecessor mechanical ventilator breath and a subsequent breath. Electrical stimulation pulses are delivered at a preset pulse rate within the designated breath. The selection of the designated stimulation breath count may be a simple ratio, such as a selected stimulation breaths occurring every other breath (1:2), occurring once every four breaths (1:4), or occurring once every 20 breaths (1:20).

Some embodiments of the control console 110 can advantageously communicate with the mechanical ventilator so that the control console 110 can receive input that is useful in assessing the patient's work of breathing and other breath characteristics. For example, a ventilator data cable 205 can provide data communication between the control console 110 and a control console of the mechanical ventilator 200 during ventilated breaths of the patient 105 (or, alternatively, the data communication connection between the control console 110 and the ventilator 200 can be a wireless connection using Bluetooth data communication or a wireless network connection). The control console 110 may receive data from the ventilator 200 indicative of the type of ventilator breath that is being output the patient. In this embodiment, the ventilator 200 can be configured to deliver flow-controlled ventilation (FCV) breaths, pressure-controlled ventilation (PCV) breaths, or a combination of both. The control console 110 can receive data from the ventilator 200 that indicates the type of ventilator breath, which in turns facilitates an interpretation of the flow measurement trace and the pressure measurement trace detected during each breath and depicted on the touchscreen interface 112 of the control console 110. In some embodiments where the control console 110 receives data communication from the ventilator 200 (e.g., via the cable 205 or a wireless data connection), the control console 110 can receive the sensed breathing characteristics (e.g., pressure, flow direction, volume, and the like) from the ventilator 200 rather than a wye sensor 120. Optionally, in such circumstances, the system 10 can operate with or without a separate wye sensor 120 connected directly to the control console 110.

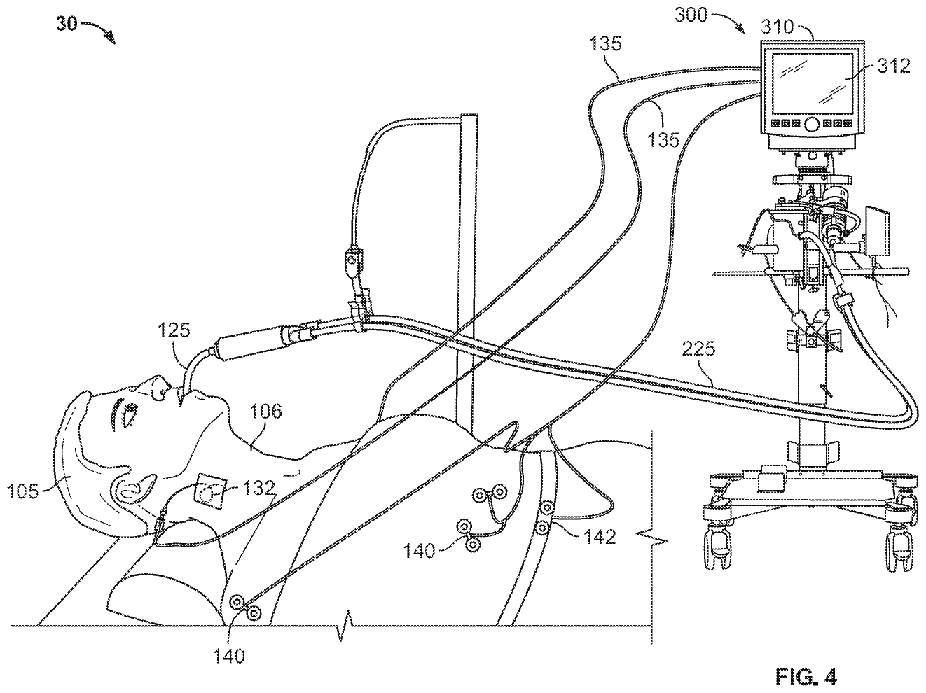

Referring now to FIG. 4, some alternative embodiments of a system 30 for diaphragm stimulation may include an improved mechanical ventilator 300 that incorporates the control of the phrenic nerve stimulation into the control console of the ventilator 300. For example, the features and operations of the control console 110 (FIGS. 1-3) described herein can be implemented in a control console 310 of the mechanical ventilator 300, and the flow detection operations of the wye sensor 120 (FIGS. 1-3) can also be performed by one or more flow sensors housed within the mechanical ventilator 300. The control console 310 of the mechanical ventilator 300 can include an improved user interface 312 that includes, in addition to control screens for setting and monitoring mechanical ventilation of the patient, further display graphics and user input fields for controlling phrenic nerve stimulation and outputting detected characteristics of the stimulated breaths (and, optionally, a work of breathing value calculated by the control console 310 of the ventilator 300 during each stimulated breath). The control console 310 of improved mechanical ventilator 300 can include ports for connection with the lead assembly 130, which includes the one or more percutaneous leads 132 and 134 connected to the ventilator 300 via cables 135. The lead assembly 130 operates as described above in connection with FIGS. 1-3, and an internal pulse generator housed within the control console 310 of the mechanical ventilator 300 (such that the ventilator 300 operates as the control console for both the mechanical ventilation controls and the phrenic nerve stimulation) can be configured to deliver the stimulation current to the selected electrode pair of each lead 132 and 134, to thereby capture the nearby phrenic nerve in the patient's neck 106 and activate the diaphragm during the mechanical breath delivered from the ventilator 300. Additionally, the control console 310 of the improved mechanical ventilator 300 can include ports for connection with optional EMG sensors, an optional body worn force sensor 142, or a combination thereof.

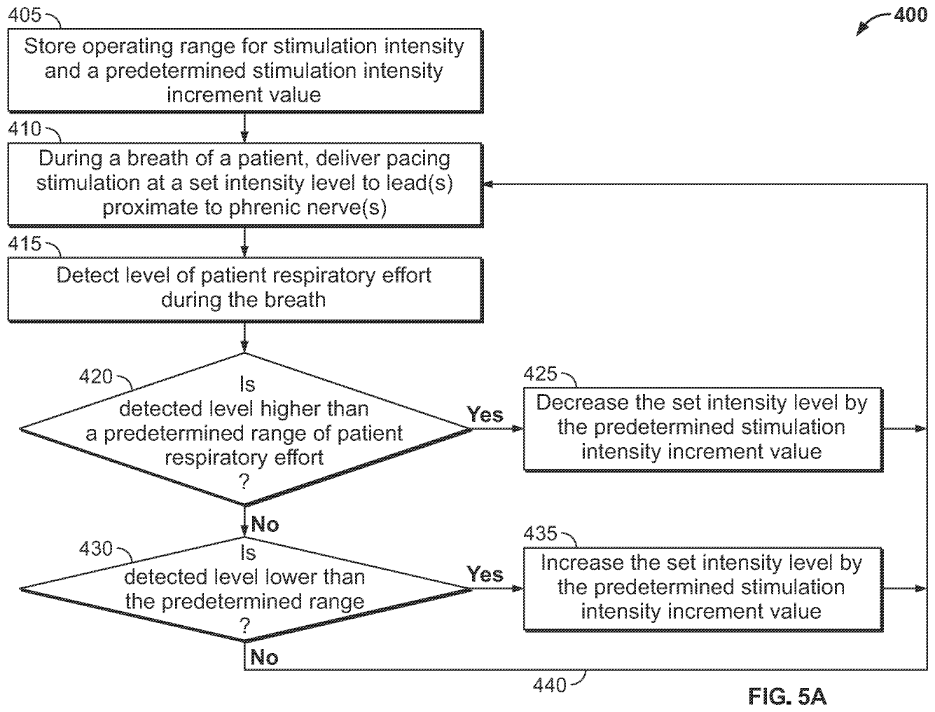

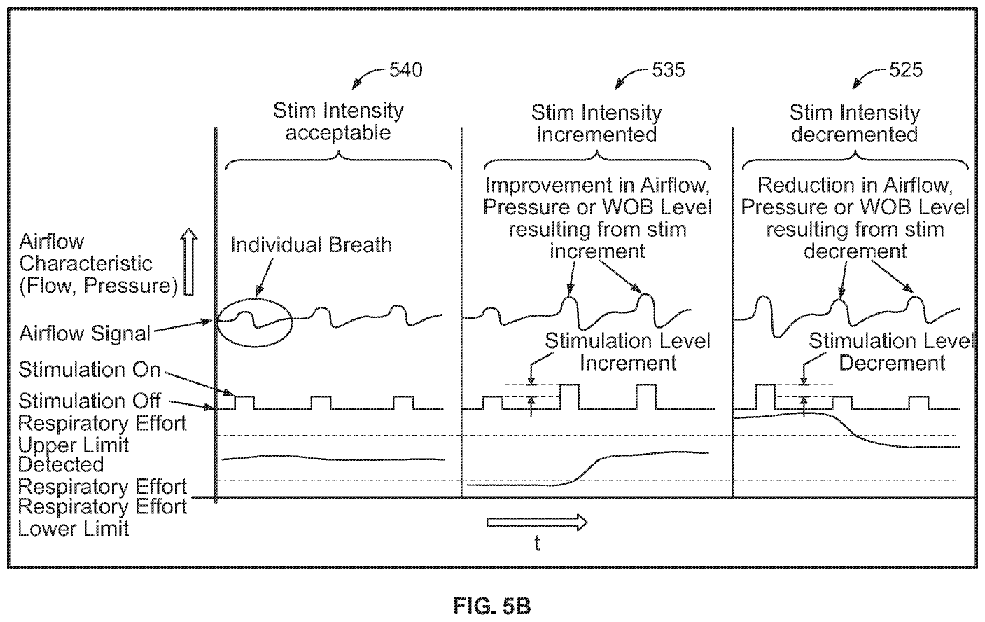

Both of the systems 10 (FIGS. 1-3D) and 30 (FIG. 4) can be implemented in a manner to perform automated stimulation control and other control features related to phrenic nerve stimulation during mechanical ventilation (including, for example, those detailed below in FIGS. 5A-12). In various versions, the control settings, user interface, and graphic summaries (e.g., including graphs of breathing characteristics during breaths) can be implemented via the control console 110 (FIGS. 1-3A) or via the control console 310 (FIG. 4).

Referring now to FIG. 5A, some embodiments of a system for diaphragm stimulation (e.g., system 10, system 30, or the like) can be implemented to achieve a method 400 that provides automatic stimulation control features. In particular examples, the system can be responsive to a respiratory effort of a patient during a breath, such as a stimulation breath during which the control console (e.g., 110 or 310) delivers an electrical stimulation signal to stimulate the patient's phrenic nerve(s). Optionally, the system can be dynamically responsive in a manner that decreases or increases the stimulation intensity level by a predetermined increment value, and the predetermined increment value can remain the same both when a detected level of patient respiratory effort is only slightly outside a predetermined range or is more significantly outside the predetermined range. In such examples, the stimulation intensity can automatically adjust over time in controlled, fixed increments, rather than significant and variable adjustments that might otherwise occur from one stimulated breath to the next stimulated breath (e.g., due to outlier measurement values for the patient respiratory effort).

As shown in FIG. 5A, the method 400 may include the operation 405 in which the control console stores an operating range for stimulation intensity, a predetermined stimulation intensity increment value, or both. In some embodiments, the stimulation intensity can be a value associated with the electrical stimulation signal that is delivered to the leads 132, 134, such as an amplitude, pulse width (PW), frequency, electrode pairs, or a combination thereof. The operating range for the for stimulation intensity that is stored by the control console can be determined manually during the electrode pair mapping session (described above) based on the diaphragm contraction threshold at the low end and the supramaximal diaphragm stimulation level at the high end (or the maximum tolerable level to the patient). In one example, electrode mapping session can identify the following characteristics for a stimulation signal and electrode pair combination (showing two configurations based on two different electrode pair combinations):

TABLE 1

| | | | Sensation | Stimulation | Diaphragm | Stimulation | Supramaximal |
|---|---|---|---|---|---|---|---|
| | | | Capture | Intensity at | Contraction | Intensity at | Diaphragm |
| | | | Threshold | WOB or | Threshold | WOB or | contraction |
| | | | Intensity | Surrogate | Intensity | Surrogate | intensity |
| Setting | | | (Amplitude, | effort signal | (Amplitude, | effort signal | (Amplitude, |
| Config. | Cathode | Anode | Pulse Width, | Low Limit | Pulse Width, | High Limit | Pulse Width, |
| # | (−) | (+) | Frequency | (0.5) | Frequency | (2.5) | Frequency |
| 1 | 1 | 3 | 0.9 mA, using | 1.3 mA, using | 1.1 mA, using | 2.3 mA, using | 2.6 mA, using |
| | | | 200us PW@ | 200us PW@ | 200us PW@ | 200us PW@ | 200us PW@ |
| | | | 25 Hz | 25 Hz | 25 Hz | 25 Hz | 25 Hz |
| 2 | 2 | 5 | 1.5 mA, using | 3.8 mA, using | 3.4 mA, using | 5.7 mA, using | 5.9 mA*, using |
| | | | 200us PW@ | 200us PW@ | 200us PW@ | 200us PW@ | 200us PW@ |
| | | | 25 Hz | 25 Hz | 25 Hz | 25 Hz | 25 Hz |

In such an example, the operating range for the for stimulation intensity stored by the control console have a low end of 1.1 mA, using 200 us PW@25 Hz and a high end of 2.6 mA, using 200 us PW@25 Hz (for configuration #1), and may have a low end of 3.4 mA, using 200 us PW@25 Hz and a high of 5.9 mA*, using 200 us PW@25 Hz (for configuration #2). In particular embodiments, the predetermined stimulation intensity increment value stored by the control console (which is used to automatically increase or decrease stimulation as detailed below) can be a proportion of the operating range, such as 10% current (mA). In this example, the increment value would be 0.15 mA (for configuration #1) and 0.25 mA (for configuration #2). Alternatively, the predetermined stimulation intensity increment value can a default value stored at the control console (e.g., 0.2 mA), which can be manually modified/selected by a user at a setting options screen of graphic user interface of the control console. Additionally, in other embodiments, the predetermined stimulation intensity increment value stored by the control console can be a characteristic of the stimulation signal other than the current amplitude, such as an increment of the pulse width (PW) or frequency value.

Still referring to FIG. 5A, the method 400 may also include the operation 410 in which, during a breath of a patient, the system delivers pacing stimulation at a set intensity level to one or more leads (e.g., leads 132, 134) proximate to phrenic nerve(s) of the patient. In such circumstances, the breath is characterized as a stimulated breath that occurs during mechanical ventilation of the patient, as described above for example, in connections with FIGS. 1-3D and 4. The set intensity level would include stimulation characteristics that fall within the range of the operating range for the for stimulation intensity stored by the control console. As previously described, set intensity level can have a stimulation intensity that is characterized by a current amplitude, pulse width (PW), frequency, or a combination thereof.

Additionally, the method 400 may also include the operation 415 in which the system detects a level of patient respiratory effort during the breath. For example, during the stimulated breath from operation 410, the system may receive sensor input (e.g., from the wye sensor 120, from the EMG sensors 140, from the patient worn sensor 142, or a combination thereof) that is indicative of a level of effort achieved by the patient's diaphragm during the stimulated breath. The sensor input can be used directly as a detected level of patient respiratory effort, or instead may be used by the control console to calculate a value for the level of patient respiratory effort. In one example, the control console can use the sensor input to calculate a Work of Breathing (WOB) value, which can be expressed as work per unit volume, for example, joules/liter, or as a work rate (power), such as joules/min. The control console can calculate the WOB during a selected breath for purposes of identifying a level of energy expended to inhale a breathing gas. In other examples, the system with the lead stimulation active may instead rely upon different characteristics for the detected level of patient respiratory effort, such as: a) peak inspiratory flow, b) mean inspiratory flow, c) peak airway pressure compared to peak predicted airway pressure, or d) mean airway pressure compared to mean predicted airway pressure. As previously described, these examples of different characteristics for the detected level of patient respiratory effort can be gathered directly from the sensor input (e.g., from the wye sensor 120, from the EMG sensors 140, from the patient worn sensor 142, or a combination thereof), without a subsequent calculation (e.g., to calculate a WOB value), to thereby provide a dynamic and possibly more prompt response from the control console (as detailed below).

For those particular embodiments in which the detected level of patient respiratory effort is a calculated WOB value, the equation of motion for respiration will be used to estimate the patient WOB in an electrically stimulated breath. In a breath without electrical stimulation the WOB should be 0 J/L because Pₘᵤₛ will be 0 cmH₂O. According to the equation of motion for the respiratory system:

Pᵥₑₙₜ+P^{mus}—elastance×volume+resistance×flow

Where elastance is a measure of the tendency of a hollow organ to recoil toward its original dimensions upon removal of a distending or compressing force. It is the reciprocal of compliance. Resistance or Airway resistance is the opposition to flow caused by the forces of friction. It is defined as the ratio of driving pressure to the rate of air flow. Elastance is measured in cmH₂O/Liter, volume in Liters, resistance in cmH₂O/Lpm and flow in Lpm. Pᵥₑₙₜ is the pressure exerted by the ventilator and Pₘᵤₛ is pressure exerted by the diaphragm muscles and both are measured in cmH₂O. This equation can be rearranged to show:

Pᵥₑₙₜ+P^{mus}—elastance×volume+resistance×flow+PEEP

P^{mus}=elastance×volume+resistance×flow+PEEP−Pᵥₑₙₜ

Where P^{vent}=P_{wye}

Work=Pressure×Volume

work=∫₀^{Vt}Pmuscles*dV (joule)
Where dV is the rate of change of volume and Vt is the tidal volume of the inspiration.

This can also be expressed as:

Work=∫ₜ₀^{t1}Pmuscles*Q dt
Where Q is the instantaneous flow, t₀ and t₁ are the start and end of inspiration.

WOB=Work/Liter=Work/Vt (joule/Liter)

The system 10, 30 can include a mechanical ventilator 200, 300 that is configured to measure, for each individual patient, respiratory mechanics properties such as static and dynamic compliance and resistance. These values can be advantageously communicated to the control console 110 in FIGS. 1-3A via the cable 205 or a wireless data connection (or measured and stored using the integrated control console 310 in FIG. 4). Additionally or alternatively, the wye sensor 120 can be configured to measure the patient's respiratory mechanics such as static and dynamic compliance and resistance, which are then stored by the control console 110, 310 for purposes of calculating the WOB value in some implementations. As described above in connection with FIGS. 1-3A and 4, the wye sensor 120 can be used to measure Pvent (Pwye) and flow at the wye (Qwye). Volume accumulation may be calculated by integrating the Qwye as the breath progress beginning at the start of inspiration and ceasing at the end of inspiration.

Referring again to FIG. 5A, the method 400 may also include operations to determine whether the detected level of patient respiratory effort (e.g., the WOB value or a sensor value as described above) falls outside a predetermined range (e.g., a selected safe range of patient effort during mechanical ventilation), and to dynamically respond accordingly by automatically adjusting the set stimulation intensity level by the pre-stored increment level. For example, in operation 420, the system can determine whether the detected level of patient respiratory effort is higher than an upper limit of a predetermined range of respiratory effort. If yes, the method 400 can respond by proceeding to operation 425 so as to decrease the set intensity level (described in operation 410) by the predetermined intensity increment value (described in operation 405). As shown in FIG. 5B, the stimulation signal can be incrementally decreased 525 in response to detecting that the level of patient respiratory effort is higher than the upper limit of the predetermined range, but later breaths may optionally remain at the same stimulation level so long as the detected level of patient respiratory effort remains within the predetermined range. (In some implementations, one or more of the traces/plots depicted in FIG. 5B can be displayed on the user interface screen of the control console 110 or 310 during treatment.) Thus, referring again FIG. 5A, operation 425 can cause the set intensity level to be decremented and then applied at the next stimulated breath as the method 400 loops back to operation 410 (which employs the decremented intensity level as the new set intensity level. In this embodiment, the increment value is pre-stored (operation 405), so the new stimulation intensity level (for the next stimulated breath) is not decreased by an amount that varies relative to how much higher the detected level of patient respiratory effort exceeds the upper limit of the predetermined range of respiratory effort. Rather, in this embodiment, the set intensity level is decremented by a preset amount regardless of whether the detected level of patient respiratory effort exceeds the upper limit by a slight amount or a more significant amount, thereby provided automated control of the stimulation intensity in a manner that avoids drastic changes in new stimulation levels from a first breath to a next stimulated breath.

Likewise, in operation 430, the system can determine whether the detected level of patient respiratory effort is lower than a lower limit of a predetermined range of respiratory effort. If yes, the method 400 can respond by proceeding to operation 435 so as to increase the set intensity level (described in operation 410) by the predetermined intensity increment value (described in operation 405). As shown in FIG. 5B, the stimulation signal can be incrementally increased 535 in response to detecting that the level of patient respiratory effort is lower than the lower limit of the predetermined range, but later breaths may optionally remain at the same stimulation level so long as the detected level of patient respiratory effort remains within the predetermined range. Thus, referring again to FIG. 5A, operation 435 can cause the set intensity level to be incremented and then applied at the next stimulated breath as the method 400 loops back to operation 410 (which employs the incrementally increased intensity level as the new set intensity level. In this embodiment, the increment value is pre-stored (operation 405), so the new stimulation intensity level (for the next stimulated breath) is not increased by an amount that varies relative to how much lower the detected level of patient respiratory effort falls below the lower limit of the predetermined range of respiratory effort. Rather, in this embodiment, the set intensity level is incrementally increased by a preset amount regardless of whether the detected level of patient respiratory effort falls below the lower limit by a slight amount or a more significant amount, again thereby providing automated control of the stimulation intensity in a manner that avoids drastic changes in new stimulation levels from a first breath to a next stimulated breath.

Still referring to FIG. 5A, the method 400 can also determine that the detected level of patient respiratory effort remains within the predetermined range (e.g. operations 420 and 430 indicate the detected level of patient respiratory effort is not above the upper limit and not below the lower limit), in which case the set intensity level can be maintained 440. As shown in FIG. 5B, the stimulation signal can be maintained 540 at the same level for a subsequent stimulated breath in response to detecting that the level of patient respiratory effort falls within the predetermined range.

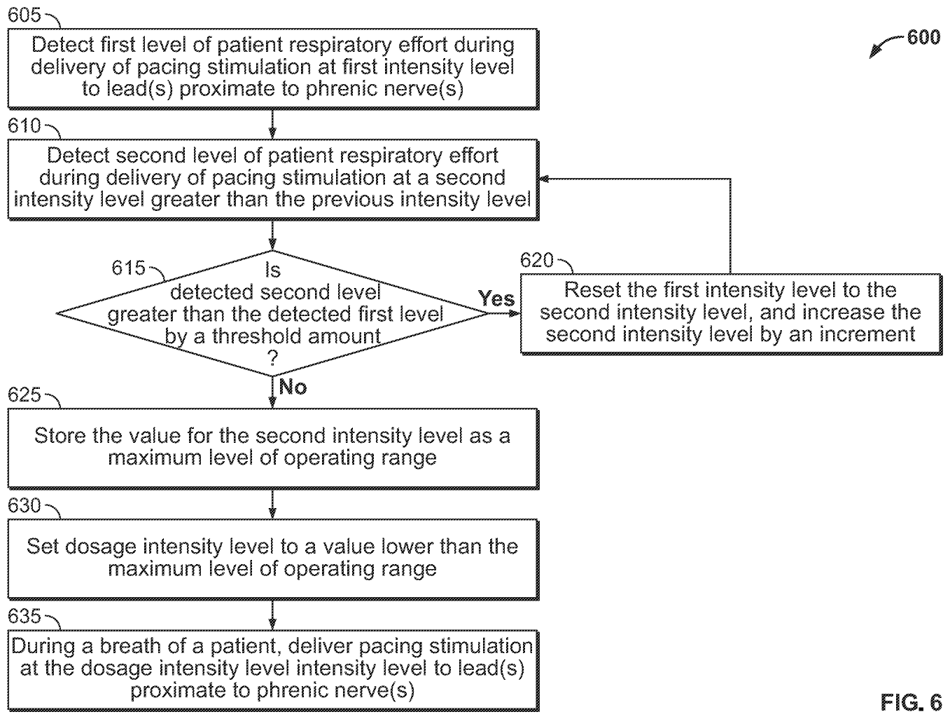

Referring now to FIG. 6, some embodiments of a system for diaphragm stimulation (e.g., system 10, system 30, or the like) can be implemented to achieve a method 600 that provides automatic stimulation dose assessment to correlate stimulation intensity to improvements in detected levels of patient respiratory effort values. In particular examples, the system can be responsive toward a respiratory effort of a patient during a breath, such as a stimulation breath during which a control console (e.g., 110 or 310) delivers an electrical stimulation signal to stimulate the patient's phrenic nerve(s). In some implementations, the method 600 be used to controllably identify a stimulation intensity level for an individual patient where the patient's respiratory effort substantially plateaus even when the stimulation level is increased. For example, in a system that calculates the WOB value for each stimulated breath, the method 600 can be used to automatically identify the stimulation intensity level where a plateau in the WOB level occurs with further increases in stimulation intensity. Such an assessment can indicate where the patient's diaphragm is maximally contacted for phrenic nerve stimulation treatment. From there, the console can store the value and report it to a user (e.g., via the graphical user interface) so that the user can select an initial level of stimulation intensity for the patient's treatment. Alternatively, the initial level of stimulation intensity can be automatically selected by the control console as a level below the level that caused maximum contraction, thereby helping avoid muscle fatigue during phrenic nerve stimulation of the patient during mechanical ventilation.

As shown in FIG. 6, the method may include operation 605 in which the system detects a first level of patient respiratory effort during delivery of pacing stimulation at a first intensity level to leads (e.g., leads 132 and 134) proximate to the patient's phrenic nerves. As previously described, set intensity level can have a stimulation intensity that is characterized by a current amplitude, pulse width (PW), frequency, or a combination thereof. Next, during a subsequent stimulated breath, the method can include operation 610 in which the system detects a second level of patient respiratory effort during delivery of pacing stimulation at a second intensity level that is greater than the previous intensity level. In operation 615, the system determines if the detected second level of patient respiratory effort is greater than the detected first level of patient respiratory effort by a threshold amount. If yes, the method 600 automatically proceeds to operation 620, in which the first intensity level is reset to a value of the second intensity level, and the second intensity level in incrementally increased by an increment. From there, the method 600 returns back to operation 610 for the next stimulated breath of the patient. This loop 610, 615, and 620 can continue until, at operation 615, the system determines that the detected second level of patient respiratory effort is not greater than the detected first level of patient respiratory effort by the threshold amount. The threshold amount can be set to a value indicative of the fact that the first and second level of patient respiratory effort are substantially close to one another (or the same), which indicates that the patient's level of respiratory effort is plateaued (or otherwise not increasing by a meaningful amount) even though the second stimulation intensity level is greater than the immediately previous intensity level during the previous stimulated breath.

As shown in FIG. 6, when operation 615 indicates that the detected second level of patient respiratory effort is not greater than the detected first level of patient respiratory effort by the threshold amount, the system proceeds to operation 625, in which the system stores the value for the second intensity level as a maximum level of operating range for the patient during the phrenic nerve stimulation treatment (e.g., during mechanical ventilation as described above). Optionally, this maximum level of operating range can also be used as an upper bound for the operating range described in connection with FIG. 5A (operation 405). As shown in FIG. 6, in operation 630, the system can be configured to automatically set a dosage intensity level to a value lower than the maximum level of operating range. In operation 635, during a selected breath of a patient, the system can deliver pacing stimulation at the dosage intensity level to leads (e.g., leads 132 and 134) proximate to the phrenic nerves. Optionally, this dosage intensity level can also be used as the set intensity level described in connection with FIG. 5A (operation 410).

During some implementations of the automated control methods described in connection with FIGS. 5A-B and 6, the control console 110, 310 can be configured to also account for migration of the leads 132 and 134 over the course of treatment of the patient. For example, the system can achieve automatic stimulation adjustment by monitoring changes in the patient respiratory effort (e.g., the calculated WOB value or a sensor value as described above). Then, in response to detecting a diminished level of patient respiratory effort on subsequent breaths, the system can automatically increase stimulation intensity (or changing electrode combinations) to maintain the target level of patient respiratory effort.

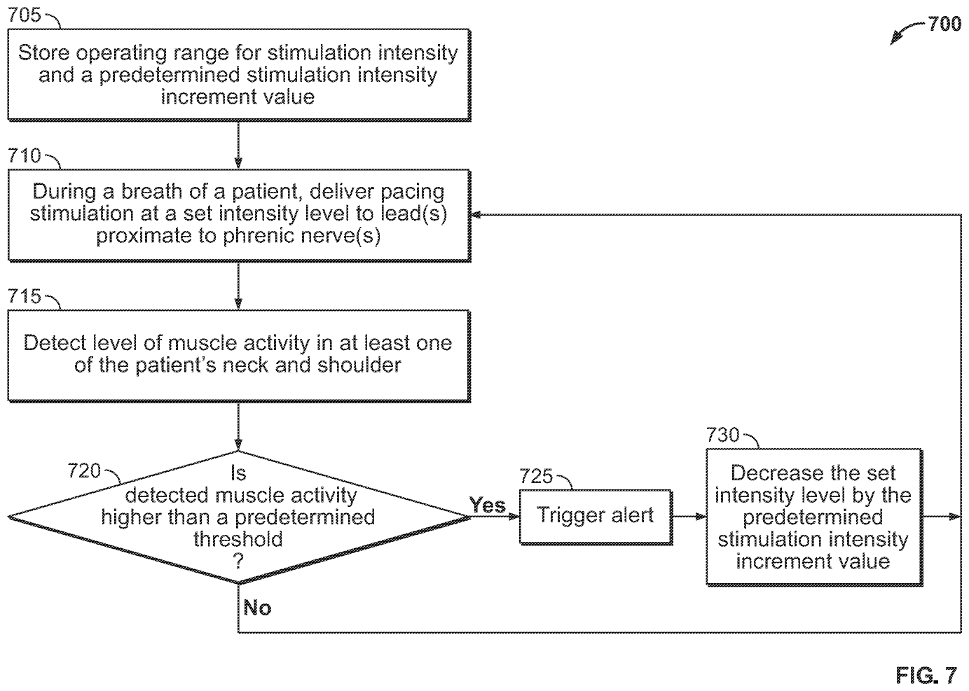

Referring now to FIG. 7, some embodiments of a system for diaphragm stimulation (e.g., system 10, system 30, or the like) can be implemented to achieve a method 700 that monitors extraneous muscle activity of a patient and responsively modifies the stimulation signal to reduce the likelihood of such extraneous muscle activity. Such a method 700 can be used, for example, a supplement to the automated control methods described above in connection with FIGS. 5A-B and 6, or can be used on a periodic basis to monitor for changes in patient positioning or migration of the leads 132, 134. In particular versions of the method, the control console 110, 310 can monitor EMG activity of the shoulder and neck muscles (e.g., using one or more EMG sensors 140 in FIGS. 1-3A and 4) and then automatically reduce the level of stimulation intensity (or change electrode combinations) to limit or eliminate extraneous stimulation in the patient's shoulder or neck muscles. In some implementations, the signals for detecting such extraneous stimulation may include those obtained using a strain sensor, ENG activity, ECG signal, accelerometry, bioimpedance or noise on the airflow signal indicative of unwanted extraneous stimulation, and/or from external sources. As shown in FIG. 7, the method 700 can include the operation 705 in which the control console stores an operating range for stimulation intensity, a predetermined stimulation intensity increment value, or both. This operation 705 can be similar to (and contemporaneous with) the operation 405 detailed above in connection with FIG. 5A. In some embodiments, the stimulation intensity can be a value associated with the electrical stimulation signal that is delivered to the leads 132, 134, such as an amplitude, pulse width (PW), frequency, electrode pairs, or a combination thereof.

Still referring to FIG. 7, the method 700 may also include the operation 710 in which, during a breath of a patient, the system delivers pacing stimulation at a set intensity level to one or more leads (e.g., leads 132, 134) proximate to phrenic nerve(s) of the patient. This operation 705 can be similar to (and contemporaneous with) the operation 410 detailed above in connection with FIG. 5. In operation 715, the system can detect a level of muscle activity beyond the activity of the patient's diaphragm muscle, such as muscle activity in the at least one of the patient's neck and shoulder. This detection 715 can occur during delivery of the stimulation signal from the pacing leads 132, 134 to the phrenic nerves (e.g., refer to FIGS. 3A-D), in which case an elevated level of muscle activity in at least one of the patient's neck or shoulder can be considered extraneous muscle activity. In operation 720, the system can determine if the detected muscle activity (in at least one of the patient's neck and shoulder) is higher than a predetermined threshold level. If not, the method can return to operation 710 (described above). If yes, the method 700 can include an operation 725, which triggers an alert via the user interface of the control console 110, 310. The alert can be audible, a visual notification, or a combination thereof. In some implementations, the alert can also provide instructions for a user to visibly monitor the patient's neck and shoulder muscles, to reposition the stimulation leads 132, 134, to select a different electrode pair for stimulation delivery, or a combination thereof. Optionally, the method 700 may also include an operation 730 in which, responsive to the determination in operation 720 that the detected muscle activity (in at least one of the patient's neck and shoulder) is higher than a predetermined threshold level, the control console can decrease the set intensity level by the predetermined threshold stimulation intensity incremental value. Thus, operation 730 can cause the set intensity level to be decremented and then applied at the next stimulated breath as the method 700 loops back to operation 710 (described above).

In further implementations of the automated control methods described in connection with FIGS. 5A-B, 6, and 7, the control console 110, 310 can include at least one stimulation lead (e.g., lead 132 or 134, or optionally, a separate sensing lead) that is configured to measure ENG (electroneurography) activity of a neighboring phrenic nerve. Phasic ENG activity may be detected and used to trigger the start of functional stimulation of the diaphragm through phrenic nerve stimulation or direct diaphragmatic stimulation. The functional stimulation pulse train may be interrupted or filtered to continue to detect native ENG to determine the point where stimulation should be stopped at end inspiration. Similarly, the level of native ENG activity may be used to control the intensity of the functional diaphragmatic stimulation. When native ENG phasic activity increases, the stimulation system could decrease the functional stimulation levels to prevent excessive diaphragmatic contraction. As detected levels of native phasic ENG decreases, the functional stimulation intensity may be increased to maintain target levels of diaphragmatic stimulation (e.g., as measured through WOB or a surrogate signal). The native ENG activity may be assessed on non-stimulated breaths to avoid interference from the stimulation signal or the stimulation signal might have a notch where native ENG activity may be assessed.

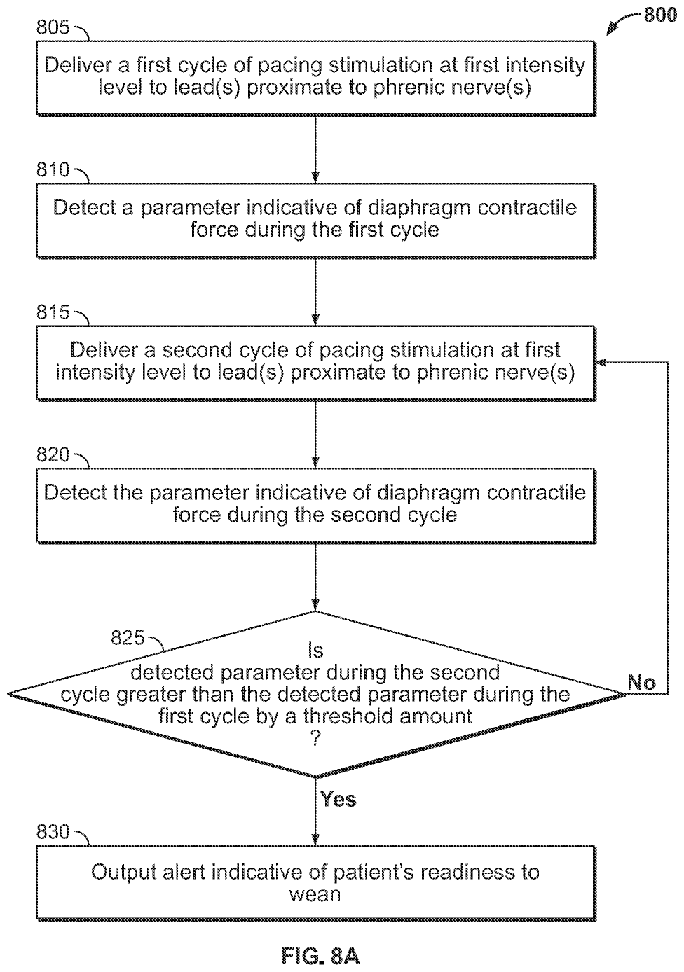

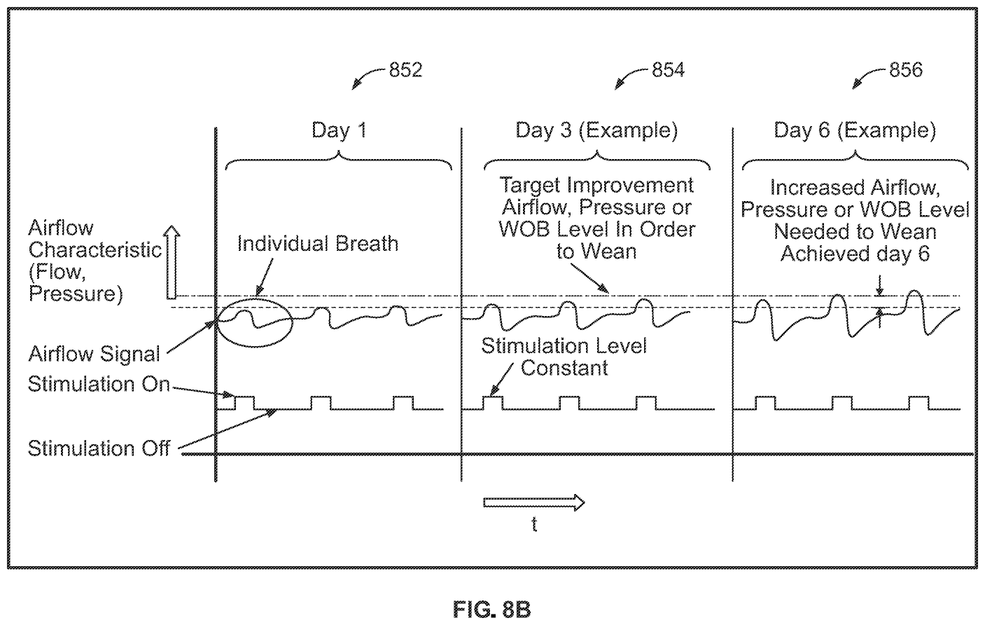

Referring now to FIGS. 8A-B, some embodiments of a system for diaphragm stimulation (e.g., system 10, system 30, or the like) can be implemented to achieve a method 800 that detects when the patient is ready to wean from mechanical ventilation and that provides a notification of such to the user. Such a method 800 can be used, for example, as a supplement to the automated control methods described above in connection with FIGS. 5A-B, 6, and 7, or can be implemented on a periodic basis to monitor for changes in patient condition. In particular versions of the method 800, the system can identify when improvement in diaphragm contractile force indicative of readiness to wean has been met, such as by monitoring improvements of the detected level of patient respiratory effort (e.g., the calculated WOB value or a sensor value as described above), which can be monitored over time (e.g., over a period of days 852, 854, and 856 depicted in FIG. 8B). Optionally, as shown in FIG. 8A, the method 800 can include an operation to alert the user when increases to the detected level of patient respiratory effort reach a predetermined target value, which can indicate that muscle conditioning of the diaphragm reached the point the diaphragm has regained enough of its original strength and weaning from the mechanical ventilator may be attempted. In some embodiments, the system can also employ an effort belt (refer to body-worn sensor 142 in FIGS. 1-3A and 4), diaphragmatic EMG (refer to diaphragmatic EMG sensor 140 in FIGS. 1-3A and 4), or other sensor indicative of respiratory effort may provide sensor feedback to the control console to indicate that acceptable improvements in diaphragmatic health have occurred, thereby providing data and notifications to the user that allow for successful weaning from of the patient from mechanical ventilation.

As shown in FIG. 8A, the method 800 can include operation 805 in which the system delivers a first cycle of pacing stimulation at a first intensity level to pacing leads (e.g., leads 132 and 134) proximate to phrenic nerves. In operation 810, the method includes detecting a parameter indicative of diaphragm contractile force during the first cycle. As previously described, parameter can be a detected level of patient respiratory effort (e.g., the calculated WOB value or a sensor value as described above). Additionally or alternatively, the parameter can be a sensor value from an effort belt (refer to body-worn sensor 142 in FIGS. 1-3A and 4), diaphragmatic EMG (refer to diaphragmatic EMG sensor 140 in FIGS. 1-3A and 4), or a combination thereof that provides sensor feedback to the control console indicative of the diaphragm contractile force. In operation 815, the system delivers a second cycle of pacing stimulation at the (same) first intensity level to pacing leads (e.g., leads 132 and 134) proximate to phrenic nerves. In some implementations, the second cycle occurs 1-3 days after the first cycle. In operation 820, the method includes detecting the parameter indicative of diaphragm contractile force during the second cycle. In operation 825, the method includes comparing the detected parameter that was detected during the second cycle and during the first cycle. For example, the system can determine in operation 825 whether the detected parameter during the second cycle is greater than the detected parameter during the first cycle by a threshold amount. Alternatively, the system can determine in whether the detected parameter during the second cycle is both greater than the detected parameter during the first cycle and greater than a targeted value indicative of the patient's readiness to wean from mechanical ventilation.

If the determination from operation 825 is no, the method 800 can loop back to operation 815 so as to evaluate a subsequent cycle of pacing stimulation at the first intensity level. If the determination from operation 825 is yes, the method 800 can proceed to operation 830 in which the control console outputs from the graphic user interface an alert indicative of the patient's readiness to wean from mechanical ventilation. As previously described, the alert can be audible, a visual notification, or a combination thereof.

Additionally or alternatively, the system described above in connection with FIGS. 8A-B can be configured identify improvement in diaphragm contractile force by monitoring improvements of the detected level of patient respiratory effort (e.g., the calculated WOB value or a sensor value as described above) over time for purposes of identifying when increases in those detected levels begin to diminish. In such circumstances, the detected level of patient respiratory effort may begin to plateau (e.g., over a period of 1-3 days), thereby indicating that muscle conditioning of the diaphragm has reached the point of diminishing returns. In response to detecting such a circumstance, the graphic user interface of the control console 110, 310 can display a plotted curve of the values for the detected level of patient respiratory effort (over a set of cycles occurring on different days), which can alert the user that the patient's diaphragm has regained its original strength and weaning may be attempted.

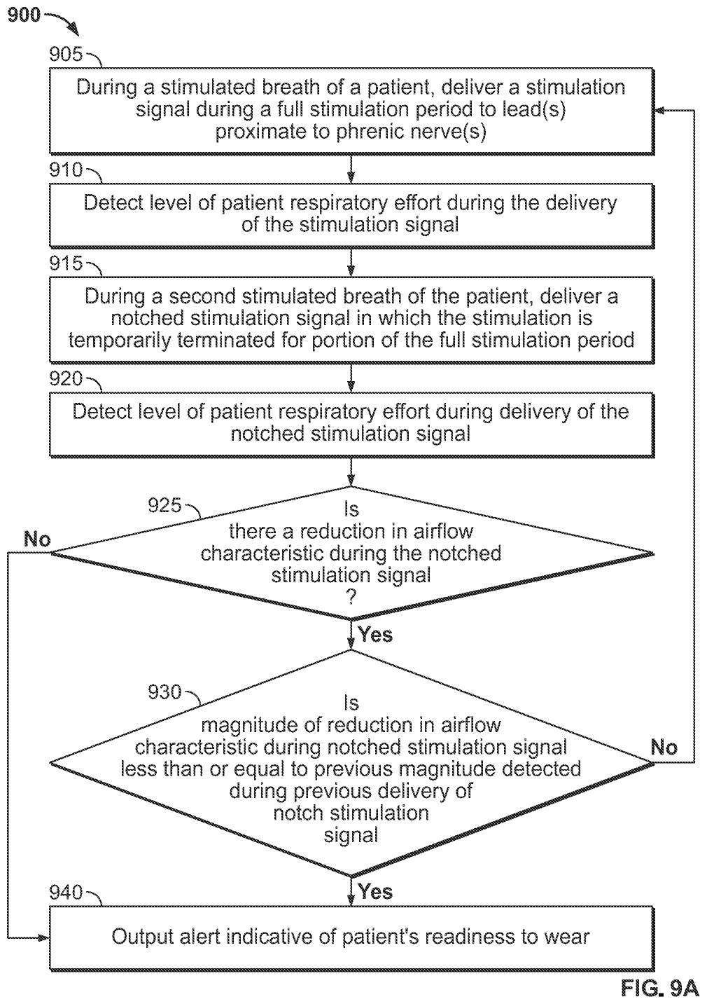

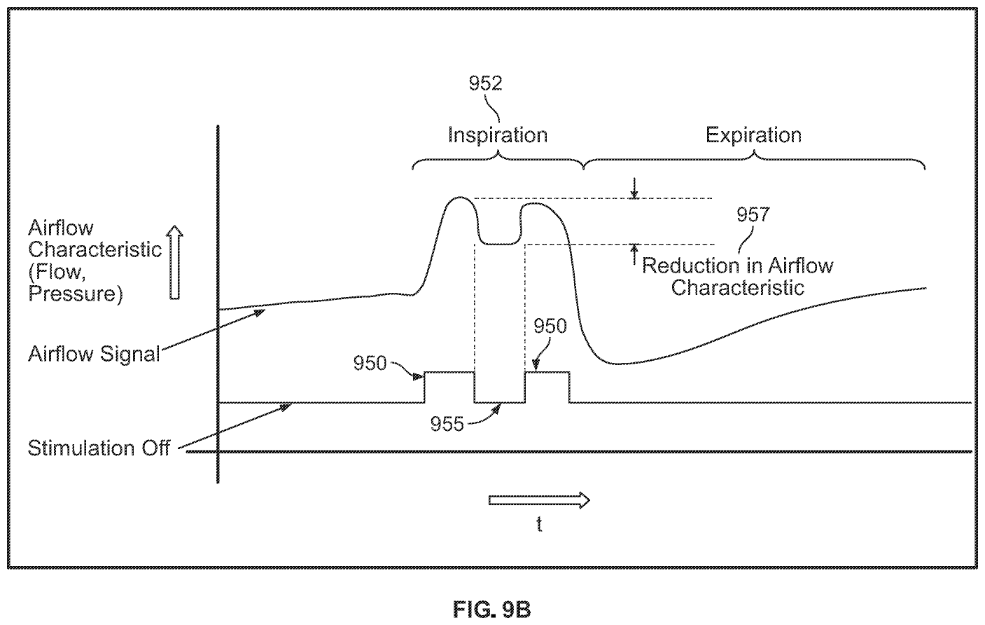

Referring now to FIGS. 9A-B, some embodiments of a system for diaphragm stimulation (e.g., system 10, system 30, or the like) can be implemented to achieve a method 900 that provides a notched pulse train delivery of phrenic nerve stimulation, which can be used to accurately assess instantaneous improvements in a patient's detected levels of respiratory effort (e.g., the calculated WOB value or a sensor value as described above). For example, the notched pulse train delivery can be output to the leads 132, 134 in a manner so that the stimulation signal is temporarily terminated for a proportion of the inspiratory period. The instantaneous change in the detected level of patient respiratory effort (e.g., the calculated WOB value or a sensor value indicative of an airflow characteristic as described above) during this notched assessment could be monitored over time to identify when improvements to the patient's detected level of respiratory effort are diminishing (e.g. diaphragm strength is approaching a sufficiency to wean from mechanical ventilation). As such, the method 900 can advantageously identify when the patient is ready to wean from mechanical ventilation and provide a notification of such to the user. Such a method 900 can be used, for example, as a supplement to the automated control methods described above in connection with FIGS. 5A-B, 6, and 7, or can be implemented on a periodic basis to monitor for changes in patient condition.

As shown in FIG. 9A, some embodiments of the method 900 can include an operation 905 in which, during a stimulated breath of a patient, the control console delivers pacing stimulation signal during a full stimulation period to one or more leads (e.g., leads 132, 134) proximate to phrenic nerve(s) of the patient. The full stimulation period can match with the inspiratory phase of the patient's breath, or be a selected portion thereof. In this embodiment of the operation 905, the pacing stimulation signal is not notched (or otherwise temporarily terminated during an intermediate portion of the stimulation period), and instead the stimulation pulses of the electrical stimulation signal are delivery consistently throughout the full stimulation period of the stimulated breath. In operation 910, the control console detects a level of patient respiratory effort during the stimulated breath. As previously described, the control console may receive sensor input (e.g., from the wye sensor 120, from the EMG sensors 140, from the patient worn sensor 142, or a combination thereof) that is indicative of a level of effort achieved by the patient's diaphragm during the stimulated breath. The sensor input can be used directly as a detected level of patient respiratory effort, or instead may be used by the control console to calculate a value for the level of patient respiratory effort (e.g., WOB). Method 900 can be implemented to perform many cycles of operations 905 and 910 before periodically (e.g., several times per day) proceeding to operation 915.

In operation 915, the control console can deliver a notch stimulation signal to the pacing leads during a subsequent stimulated breath, in which the stimulation signal is temporarily terminated for a portion of the full stimulation period (e.g., an intermediate portion of the inspiratory period of the stimulated breath). For example, as shown in FIG. 9B, the notched stimulation signal 950 can be aligned with the inspiratory period 952 of the patient during the stimulated breath, but the stimulation signal includes a “notch” 955 where the stimulation pulses of the stimulation signal are temporarily terminated during an intermediate portion of the stimulated breath. (In some implementations, one or more of the traces/plots depicted in FIG. 9B can be displayed on the user interface screen of the control console 110 or 310 during treatment.) In response to the temporary termination of the stimulation pulses, the system can detect (e.g., via the wye sensor 120) a reduction in an airflow characteristic 957, such as an instantaneous change in the detected flow volume or pressure. As described below, the magnitude of this reduction in the airflow characteristic can be monitored over time (e.g., when operation 915 of method 900 is performed several times during a day or over a period of days) to provide an objective indicator of when measurable improvements to the patient's level of respiratory effort are plateauing (e.g., because the patient's diaphragm has reached sufficient strength to be ready to wean from the mechanical ventilator).

Referring again to FIG. 9A, the method can include operation 920 the control console detects the level of patient respiratory effort during the delivery of the notched stimulation signal (operation 915). In operation 925, the method 900 detects if there is a reduction in the airflow characteristic (e.g., from the wye sensor 120 detecting the flow volume or pressure during the inspiratory phase) during delivery of the notched stimulation signal (operation 915). The reduction in the airflow characteristic can be identified by comparing the magnitude of the airflow characteristic measured during the notched stimulation signal to the previous magnitude of the same airflow characteristic measured during the regular stimulation signal previously delivered throughout the full stimulation period. For example, the system may detect there is no reduction in the airflow characteristic where a magnitude of any reduction in the airflow characteristic during delivery of the notched stimulation signal is less than a predetermined low threshold. If there is no reduction detected at operation 925, the system can proceed to operation 940 to alert the user that muscle conditioning of the diaphragm reached the point the diaphragm has regained enough of its original strength and weaning from the mechanical ventilator may be attempted. If there is a reduction detected at operation 925, the system can proceed to operation 930 where the magnitude of the reduction is compared to a previously measured magnitude of a reduction in the airflow characteristic during a previous delivery of a notched stimulation signal (e.g., during a previous cycle of method 900 when a previous instance of operation 915 was performed). If the detected reduction of the airflow characteristic during the most recent delivery of the notched stimulation signal is less than or equal to the previous magnitude of the reduction in the airflow characteristic during a previous delivery of a notched stimulation signal, the system proceeds to operation 940 to alert the user that muscle conditioning of the diaphragm reached the point the diaphragm has regained enough of its original strength and weaning from the mechanical ventilator may be attempted. If not, the method 900 can loop back to operation 905 for continued treatment of the patient during mechanical ventilation.

In further implementations of methods described in connection with FIGS. 8A-B and 9A-B, the control console 110, 310 can include at least one lead (e.g., lead 132 or 134, or optionally, a separate sensing lead) that is configured to measure ENG activity of the phrenic nerve adjacent to the lead. In those embodiments, measurement of ENG activity can be compared to the resulting diaphragm contraction level as an indication of the improved state of the diaphragm resulting from phrenic nerve stimulation training. The diaphragm health may be monitored periodically by measuring detected increases to the patient's level of respiratory effort (e.g., the calculated WOB value or a sensor value as described above) while the stimulation signal is terminated (e.g., temporarily terminated during a notched stimulation signal, as described above) to identify when these parameters have reached a specific target value or reached a plateau, indicating the targeted benefit of stimulation therapy has been reached and the patient's diaphragm strength has sufficiently increased. In addition, an effort belt (e.g., body worn sensor 142, diaphragmatic EMG sensors (e.g., sensors 140), or other sensor indicative of respiratory effort could be used to provide feedback to the control console, which may compare ENG activity as a measurement of improvements or degradations diaphragmatic health.

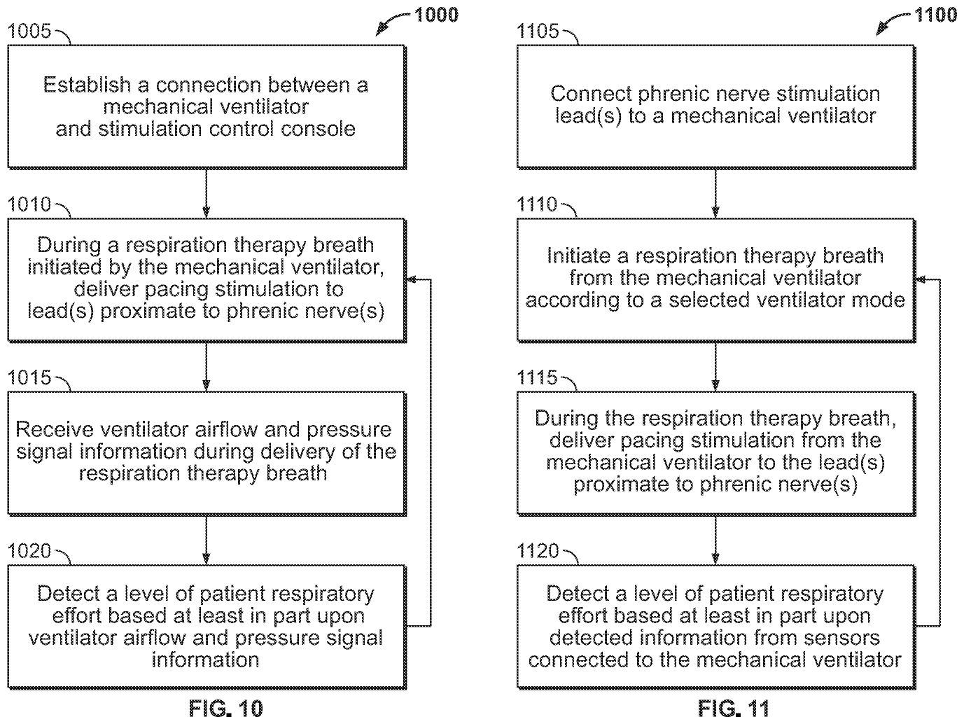

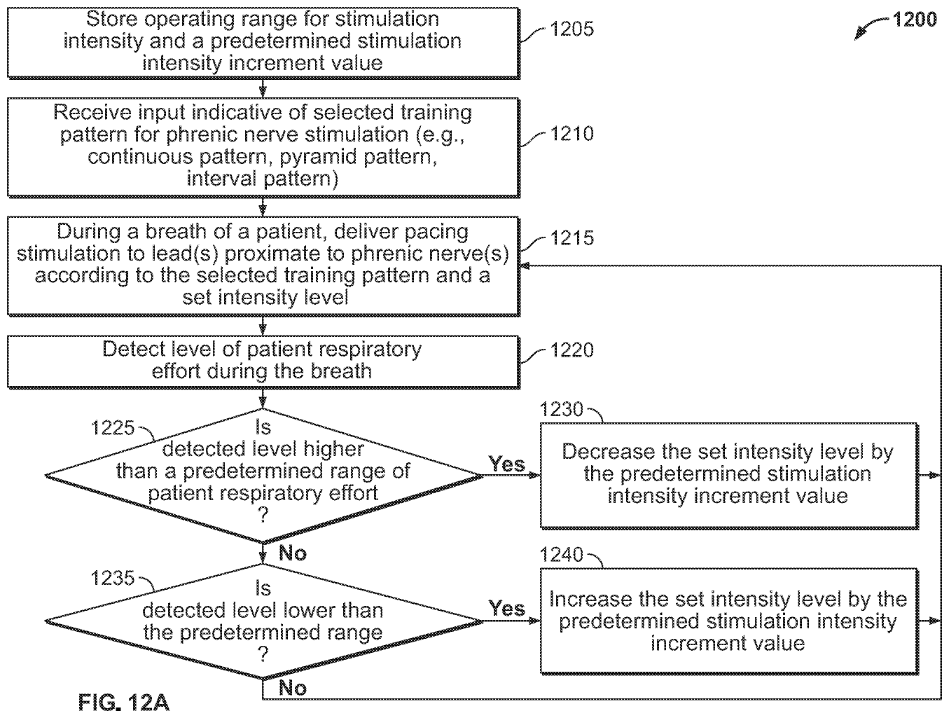

Referring now to FIGS. 10-11, some embodiments of a system for diaphragm stimulation (e.g., system 10, system 30, or the like) can be implemented to achieve data communication between a stimulation control console and a control console of a mechanical ventilator (e.g., ventilator 200) or to achieve or phrenic nerve stimulation control directly from an improved mechanical ventilator (e.g., ventilator 300). In such circumstances, the system can perform improved automated control of the phrenic nerve stimulation, for example, using the method 1000 or 1100 described in FIGS. 10-11. As previously described in connection with FIGS. 1-3A, some embodiments of the control console 110 can advantageously receive data from the ventilator 200 that is useful in assessing the patient's work of breathing and other breath characteristics. Additionally, the control console 110 may communicate data back to the ventilator 200. In one example, the ventilator data cable 205 can provide data communication between the control console 110, and the control console 110 may receive data from the ventilator 200 indicative of the type of ventilator breath that is being delivered to the patient. The control console 110 can receive data from the ventilator 200 that indicates the type of ventilator breath, which in turns facilitates an interpretation of the flow measurement trace and the pressure measurement trace detected by the wye sensor 120 during each breath and depicted on the touchscreen interface 112 of the control console 110. Additionally or alternatively, the control console 110 may receive data from the ventilator 200 indicative of the patient-specific measurements of respiratory mechanics such as the patient's lung compliance and resistance. As previously described in connection with FIG. 4, some version of an improved mechanical ventilator 300 can include a control console 310 for dictating both the control of mechanical ventilation and the control of the phrenic nerve stimulation. In such cases, the control console 310 of the mechanical ventilator 300 can include an improved user interface 312 that includes (i) control screens/user input fields for setting and monitoring mechanical ventilation of the patient and (ii) control screens/user input fields setting and monitoring the phrenic nerve stimulation and outputting detected characteristics of the stimulated breaths.

As shown in FIG. 10, some embodiments of a method 1000 can be used during data communication between a phrenic nerve stimulation console (e.g., control console 110) and a mechanical ventilator (e.g., ventilator 200). The method can include operation 1005 to establish a connection between a mechanical ventilator and a stimulation control console. As previously described, the control console may be connected to a mechanical ventilator using a cable (e.g., cable 205) or a wireless data connection such as Bluetooth. In operation 1010, the system can deliver pacing stimulation to one or more leads proximate to one or more phrenic nerves during respiration therapy initiated by the mechanical ventilator. The method 1000 can also include operation 1015 in which the control console receives ventilator airflow and pressure signal information during delivery of the respiration therapy breath. Optionally, the data connection between the stimulation control console and the ventilator can be used to transfer ventilator settings to the control console. For example, the control console can receive settings information from the ventilator indicative of the type of ventilator breath that is being output the patient (e.g., flow-controlled ventilation (FCV) breaths, pressure-controlled ventilation (PCV) breaths, or a combination of both). In operation 1020, the system can detect a level of patient respiratory effort (e.g., calculate the WOB value or using sensor measurements directly as described above) based at least in part upon the data received from the ventilator via the data connection. For example, through the data connection (operation 1005), ventilator airflow and pressure signals may be sent to the stimulation control console for purposes of detecting a level of patient respiratory effort, which can be used to automatically control stimulation intensity, determine readiness to wean or determine if unwanted extraneous stimulation exists (as detailed in the example methods above). Additionally, the control console 110 can receive the settings information from the ventilator 200 and then use such information for interpreting the flow measurement trace and the pressure measurement trace detected during each breath and depicted on the touchscreen interface of the control console.

As shown in FIG. 11, some embodiments of a method 1100 can be used with an improved mechanical ventilator (e.g., ventilator 300) having a control console that operates both the control of mechanical ventilation and the control of the phrenic nerve stimulation. The method 1100 can include operation 1105 in which one or more phrenic nerve stimulation leads (e.g., leads 132 and 134) are connected to mating connector ports of a mechanical ventilator. In operation 1110, the system can initiate a respiration therapy breath from the mechanical ventilator according to a selected ventilator mode. In operation 1115, during the respiration therapy breath, the system can deliver pacing stimulation from the mechanical ventilator to the one or more leads proximate to one or more phrenic nerves. In operation 1120, the system can detect a level of patient respiratory effort (e.g., calculate the WOB value or using sensor measurements directly as described above) based at least in part upon the information sensed by the ventilator (e.g., one or more flow sensors housed within the mechanical ventilator). For example, the ventilator can be equipped to detect airflow and pressure signals during the mechanical ventilation of the patient, and the sensor information can be used by ventilator's control console for purposes of detecting a level of patient respiratory effort, which can be used to automatically control stimulation intensity, determine readiness to wean or determine if unwanted extraneous stimulation exists (as detailed in the example methods above).

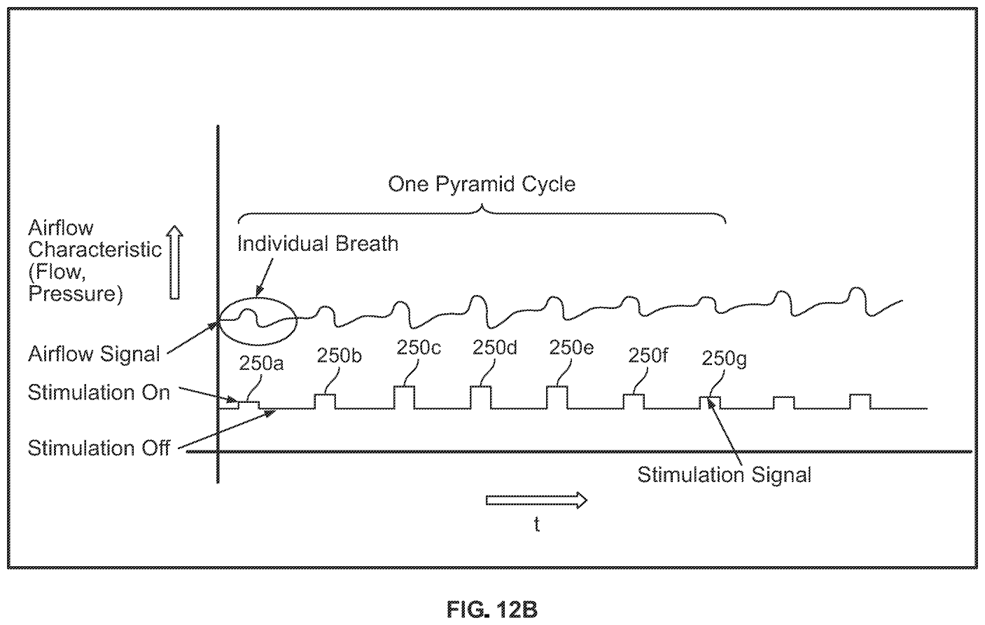

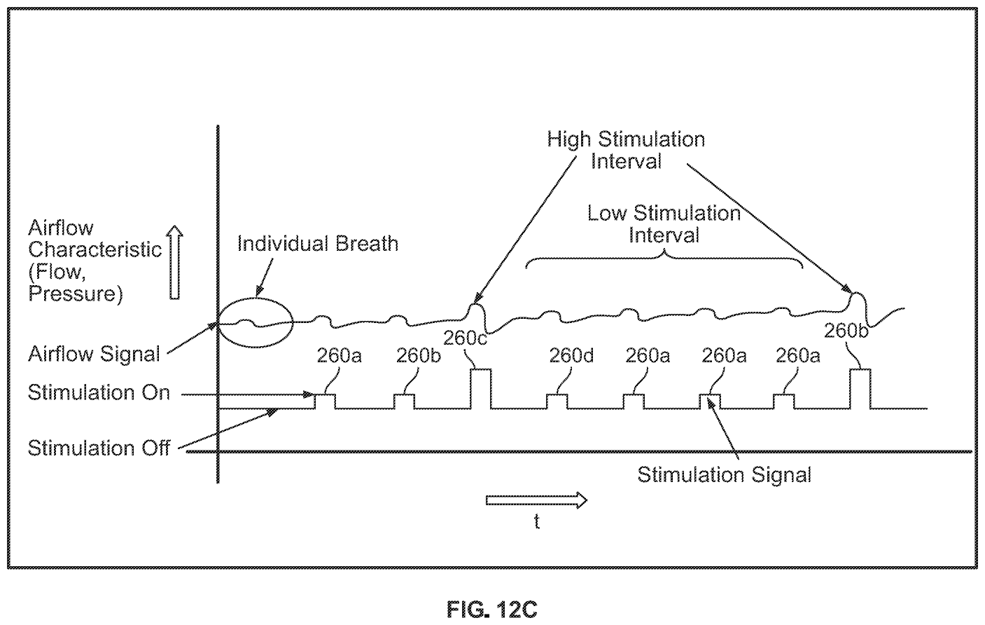

Referring now to FIGS. 12A-C, some embodiments of method 1200 can be implemented by a system for diaphragm stimulation (e.g., system 10, system 30, or the like) to achieve phrenic nerve stimulation training patterns, which can be automatically selected by a control console or selected by a user for purposes of improving the therapeutic benefit of strengthening a patient's diaphragm during phrenic nerve stimulation therapy. In particular embodiments, the control console can be implemented to deliver stimulation in one of a variety of selectable patterns. Such a system can improve a rate at which the patient's diaphragm strength is increased and, in some embodiments, can facilitate an improved maximum level of diaphragm strength at the end of treatment. As shown in FIG. 12A, the method 1200 can include an operation 1205 in which the control console stores an operating range for stimulation intensity, a predetermined stimulation intensity increment value, or both. As previously described above in connection with FIG. 4, the stimulation intensity can be a value associated with the electrical stimulation signal that is delivered to the leads 132, 134, such as an amplitude, pulse width (PW), frequency, electrode pairs, or a combination thereof. The operating range for the for stimulation intensity that is stored by the control console can be determined manually during the electrode pair mapping session (described above) based on the diaphragm contraction threshold at the low end and the supramaximal diaphragm stimulation level at the high end (or the maximum tolerable level to the patient). The method 1200 can also include operation 1210 in the control console receives input indicative of a selected training pattern for phrenic nerve stimulation. For example, the user interface of the control console (e.g., console 110 or 310) can present selectable training options, such as a continuous pattern, a pyramid pattern, or an interval pattern, and a user can select the option for a particular patient. In operation 1215, during a stimulated breath of a patient, the system can deliver pacing stimulation to one or more leads (e.g., leads 132 and 134) proximate to phrenic nerves according to the selected training pattern and the set intensity level (within the operating range described in operation 1205). For example, as shown in FIG. 12B, the training pattern implemented for a particular patient may be in the form of a “pyramid” training pattern 250a-250g, in which the stimulation delivered may start at a relatively low level 250a, ramping up to a peak value 250d, and then ramping back down to the low level 250g at the end of the session for the stimulation therapy. Incrementing and decrementing of the stimulation might be done on a breath-by-breath basis or might remain constant for several breaths or more before changing to the next level. The “pyramid” pattern might repeat until the entire therapy session is complete. In another example, as shown in FIG. 12C, the training pattern implemented for a particular patient may be in the form of an “interval” training pattern where lower stimulation intensity 260a is delivered for a number of breaths providing a nominal level of diaphragmatic contraction, followed by one or more higher intensity stimulation 260b during stimulated breath(s) nearer the peak contraction of the diaphragm. The lower level stimulation 260a might then return for a series, resulting in an “interval” training regimen. In further examples, the training pattern implemented for a particular patient may be in the form of: a higher stimulation intensity level that is applied for a shorter duration (e.g., shorter than a regular session time), an extended stimulation duration where the normal intensity level is applied for a duration that is greater than a regular session time, or an increased rate training session in which the frequency of stimulation breaths is increased during a shorter training duration (e.g., shorter than a regular session time).

Referring again to FIG. 12A, the method 1200 can include the operation 1220 in which the system detects a level of patient respiratory effort during the stimulated breath (e.g., delivery of pacing stimulation). The system can use this detected level of patient respiratory effort to provide adaptive training patterns for the phrenic nerve stimulation therapy. In some example, the control console can be configured to automatically adapt the set intensity level of the pacing stimulation during the training pattern, for example, by automatically adjusting the magnitude of the electrical current for each stimulation signal within the training pattern.

For example, the method 1200 may be used determine whether the detected level of patient respiratory effort (e.g., the WOB value or a sensor value as described above) falls outside a predetermined range (e.g., a selected safe range of patient effort during mechanical ventilation), and to dynamically respond accordingly by automatically adjusting the set stimulation intensity level across all stimulations for the training pattern. For example, in operation 1225, the system can determine whether the detected level of patient respiratory effort is higher than an upper limit of a predetermined range of respiratory effort. If yes, the method 1200 can respond by proceeding to operation 1230 so as to decrease the set intensity level (described in operation 1215) by the predetermined intensity increment value (described in operation 1205), which thereby decrements the stimulation intensity for each stimulated breath in the selected training pattern. In this embodiment, the increment value is pre-stored (operation 1205), so the new stimulation intensity level (for the next stimulated breath) is not decreased by a variable amount. Rather, in this embodiment, the set intensity level is decremented by a preset amount regardless of whether the detected level of patient respiratory effort exceeds the upper limit by a slight amount or a more significant amount, thereby provided automated control of the stimulation intensity in a manner that avoids drastic changes in new stimulation levels from a first breath to a next stimulated breath.

Likewise, in operation 1235, the system can determine whether the detected level of patient respiratory effort is lower than a lower limit of a predetermined range of respiratory effort. If yes, the method 1200 can respond by proceeding to operation 1240 so as to increase the set intensity level (described in operation 1215) by the predetermined intensity increment value (described in operation 1205), which thereby incrementally increases the stimulation intensity for each stimulated breath in the selected training pattern. Here again, in this embodiment, the increment value is pre-stored (operation 1205), so the set intensity level is incrementally increased by a preset amount regardless of whether the detected level of patient respiratory effort falls below the lower limit by a slight amount or a more significant amount. In doing so, the system can provide adaptive training cycles for the patient in a manner that avoids drastic changes in new stimulation levels from a first breath to a next stimulated breath. Also, if the method 1200 determines that the detected level of patient respiratory effort remains within the predetermined range (e.g. operations 1225 and 1235 indicate the detected level of patient respiratory effort is not above the upper limit and not below the lower limit), in which case the training pattern need not be adapted to a different intensity and the set intensity level can be maintained.

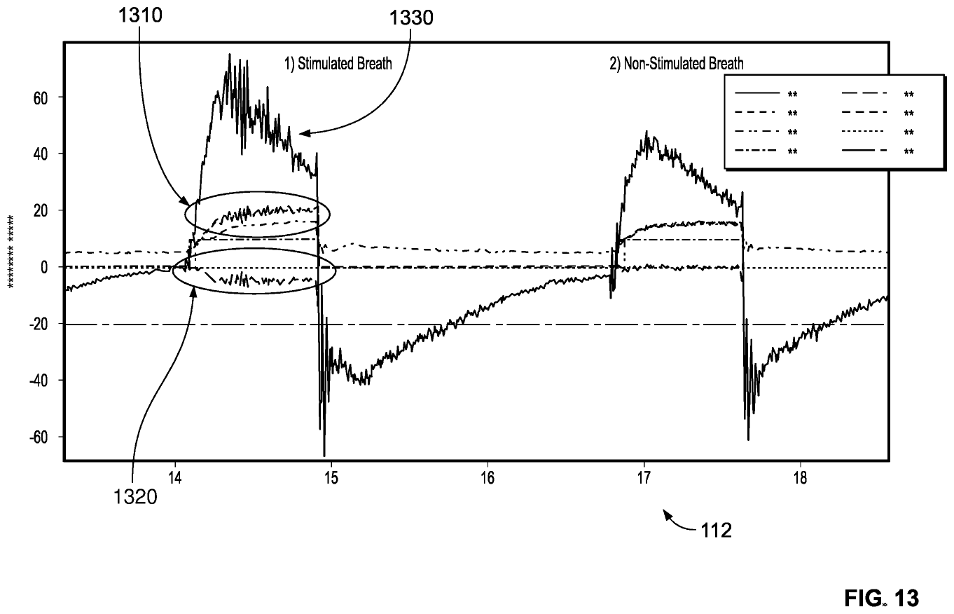

Referring now to FIG. 13, some embodiments of a system for diaphragm stimulation (e.g., system 10, system 30, or the like) can be implemented rely upon a sensor feedback for the detected level of patient respiratory effort, which can provide a dynamic control response that does not rely upon subsequent calculations for work of breathing (WOB). For example, an operator may set a pressure goal for PawPred (predicted Airway Pressure). Stimulation levels may be controlled on a sample-by-sample basis using a MD controller, or like methodology, to identify whether or not the pressure goal has been achieved. In such embodiments, the control console (FIGS. 1-3A and 4) can be configured to automatically adjust the stimulation level to meet the pressure goal. Additionally or alternatively, an operator may set a waveform shape for PawPred. A system or apparatus as characterized herein may control stimulation levels on a sample-by-sample basis using a PID controller, or like methodology, to identify whether or not the waveform goal has been achieved. In such embodiments, the control console (FIGS. 1-3A and 4) can be configured to automatically adjust the stimulation level to meet the waveform goal. Additionally or alternatively, an operator may set a negative pressure goal for PDia (diaphragm induced pressure). This arithmetically is PawMeasured (measured Airway Pressure)—PawPred. A system or apparatus as characterized herein may control stimulation levels on a sample-by-sample basis using a PID controller, or like methodology, to identify whether or not the pressure goal has been achieved. In such embodiments, the control console (FIGS. 1-3A and 4) can be configured to automatically adjust the stimulation level to meet the pressure goal. Additionally or alternatively, an operator may set negative pressure waveform shape for PDia. A system or apparatus as characterized herein may control stimulation levels on a sample-by-sample basis using a PID controller, or like methodology, to identify whether or not the waveform goal has been achieved. In such embodiments, the control console (FIGS. 1-3A and 4) can be configured to automatically adjust the stimulation level to meet the waveform goal. Additionally or alternatively, an operator may set Peak Inspiratory Flow (PIF) or Inspiratory Tidal volume (TVI) goal. A system or apparatus as characterized herein may control stimulation levels on a sample-by-sample basis using a PID controller or like methodology, to identify whether or not the goal has been achieved. In such embodiments, the control console (FIGS. 1-3A and 4) can be configured to automatically adjust the stimulation level to meet the goal. Additionally or alternatively, an operator may set waveform shape for Vaw (measured Airway Flow). A system or apparatus as characterized herein may control stimulation levels on a sample-by-sample basis using a PID controller, or like methodology, to identify whether or not the goal has been achieved. In such embodiments, the control console (FIGS. 1-3A and 4) can be configured to automatically adjust the stimulation level to meet the goal.

Refer again to FIGS. 1-4, the control console 110 (FIGS. 1-3A) or 310 (FIG. 4) includes a graphic user interface (e.g., a touchscreen and, optionally, mechanical switches for user input), along with a processor and computer-readable memory positioned within the housing that are configured to control stimulation of the patient's diaphragm according to the implementations detailed above. The memory housed within the control console 110 or 310 is capable of providing mass storage for the system 100 or 300, and may be in the form of a computer-readable medium, such as a solid state memory device or a hard disk device. In some options, the control console 110 or 310 can include a high-speed interface connecting to the memory and to multiple high-speed expansion ports, and at least one port to receive an external memory device (e.g., a USB drive or external hard drive). Each of the processor, the memory, the high-speed interface, the high-speed expansion ports may be interconnected using various busses, and can be mounted on a motherboard. The processor can process instructions for execution of the stimulation control operations and other tasks detailed above, including instructions stored in the memory to display graphical information for the graphic user interface 112 or 312 (detailed above). Accordingly, certain embodiments are directed to a computer program product (e.g., nonvolatile memory device), which includes a machine or computer-readable medium having stored thereon instructions which may be executed by a computer (or other electronic device) to perform these operations/activities. For instance, such a product may be implemented with circuitry otherwise operable to facilitate respiration, and may be implemented in accordance with one or more embodiments herein.

A number of embodiments have been described. Nevertheless, it will be understood that various modifications may be made without departing from the scope of the following claims. For example, multiple variations of trigger points for control may be utilized, for instance to adjust stimulation to various levels that are preset. Accordingly, other embodiments are within the scope of the following claims.

1. A phrenic nerve stimulation system, comprising:
a phrenic nerve stimulation control console configured to control a stimulation intensity of a phrenic nerve stimulation signal deliverable to at least one phrenic nerve during mechanical ventilation; and
a phrenic nerve stimulation lead assembly including at least one electrode lead positionable proximate to the at least one phrenic nerve to output the phrenic nerve stimulation signal from the phrenic nerve stimulation control console to the corresponding phrenic nerve,
wherein, in response to a detected level of patient respiratory effort being outside a predetermined range, the phrenic nerve stimulation control console controllably increments or decrements the stimulation intensity of the phrenic nerve stimulation signal by a preset increment value.

2. The system of claim 1, further comprising a breath sensor in communication with the phrenic nerve stimulation control console and being configured to couple with an air flow path of a breathing circuit during mechanical ventilation, wherein a sensor information from the breath sensor is received at the phrenic nerve stimulation control console so that the phrenic nerve stimulation control console identifies the detected level of patient respiratory effort.

3. The system of claim 1, wherein the phrenic nerve stimulation control console controllably increments the stimulation intensity of the phrenic nerve stimulation signal by the preset increment value in response to the detected level of patient respiratory effort being lower than the predetermined range.

4. The system of claim 3, wherein the phrenic nerve stimulation control console controllably decrements the stimulation intensity of the phrenic nerve stimulation signal by the preset increment value in response to the detected level of patient respiratory effort being higher than the predetermined range.

5. The system of claim 4, wherein the phrenic nerve stimulation control console includes a graphic user interface to receive user input of a selected value for the preset increment value.

6. The system of claim 5, wherein the graphic user interface of the phrenic nerve stimulation control is configured to receive a customized selection of an operating range for the stimulation intensity of the phrenic nerve stimulation signal.

7. The system of claim 1, wherein the phrenic nerve stimulation control console is configured to automatically identify a maximum level of stimulation intensity of an operating range for the stimulation intensity of the phrenic nerve stimulation signal, wherein the phrenic nerve stimulation control console controllably increments or decrements the stimulation intensity by the preset increment value so that the stimulation intensity remains within the operating range.

8. The system of claim 1, wherein the phrenic nerve stimulation control console is integrated into a console of a mechanical ventilator such that a cable of the phrenic nerve stimulation lead assembly mates with the mechanical ventilator

9. The system of claim 1, wherein the at least one electrode lead of the phrenic nerve stimulation lead assembly a first electrode lead and a second electrode lead, and each of the first and second electrode leads comprises one of: a percutaneously insertable paddle electrode lead tip having an array of electrodes along a planar surface, and multiple segmented electrodes along a cylindrical portion of a percutaneously insertable lead tip.

10. A phrenic nerve stimulation system, comprising:
a phrenic nerve stimulation control console configured to control a stimulation intensity of a phrenic nerve stimulation signal deliverable to at least one phrenic nerve during mechanical ventilation;
a phrenic nerve stimulation lead assembly including at least one electrode lead positionable proximate to the at least one phrenic nerve to output the phrenic nerve stimulation signal from the phrenic nerve stimulation control console to the corresponding phrenic nerve; and
a muscle activity sensor in communication with the phrenic nerve stimulation control console to provide feedback indicative of muscle activity in at least one of a shoulder and neck,
wherein, in response to a detected level of muscle activity being greater a predetermined threshold, the phrenic nerve stimulation control console decrements the stimulation intensity of the phrenic nerve stimulation signal by a preset increment value.

11. The system of claim 10, wherein the phrenic nerve stimulation control console includes a graphic user interface that outputs an alert in response to the detected level of muscle activity being greater a predetermined threshold.

12. The system of claim 10, further comprising a breath sensor in communication with the phrenic nerve stimulation control console and being configured to couple with an air flow path of a breathing circuit during mechanical ventilation, wherein a sensor information from the breath sensor is received at the phrenic nerve stimulation control console so that the phrenic nerve stimulation control console identifies a detected level of patient respiratory effort.

13. The system of claim 12, wherein the phrenic nerve stimulation control console controllably increments the stimulation intensity of the phrenic nerve stimulation signal by a preset increment value in response to the detected level of patient respiratory effort being lower than the predetermined range.

14. The system of claim 13, wherein the phrenic nerve stimulation control console controllably decrements the stimulation intensity of the phrenic nerve stimulation signal by the preset increment value in response to the detected level of patient respiratory effort being higher than the predetermined range.

15. The system of claim 14, wherein the phrenic nerve stimulation control console includes a graphic user interface to receive user input of a selected value for the preset increment value.

16. The system of claim 10, wherein the phrenic nerve stimulation control console is integrated into a control unit of a mechanical ventilator.

17. A method of controlling phrenic nerve stimulation, comprising:
after delivering a first cycle of phrenic nerve stimulation at a first intensity level from a phrenic nerve stimulation apparatus to at least one stimulation lead proximate to at least one phrenic nerve, delivering a second cycle of phrenic nerve stimulation at the first intensity level from the phrenic nerve stimulation apparatus to the at least one stimulation lead proximate to the at least one phrenic nerve; and
in response to detecting that a parameter indicative of diaphragm contractile force is greater during the second cycle than the first cycle, outputting an alert via a user interface of the phrenic nerve stimulation apparatus indicative of a readiness to wean from mechanical ventilation.

18. The method of claim 17, wherein the phrenic nerve stimulation apparatus includes:
a phrenic nerve stimulation control console configured to control stimulation intensity of a phrenic nerve stimulation signal deliverable to the at least one phrenic nerve during mechanical ventilation; and
a phrenic nerve stimulation lead assembly including the at least one stimulation lead positionable proximate to the at least one phrenic nerve to output the phrenic nerve stimulation signal from the phrenic nerve stimulation control console.

19. The method of claim 18, further comprising receiving breath sensor information from a breath sensor in communication with the phrenic nerve stimulation control console and being coupled with an air flow path of a breathing circuit during mechanical ventilation, and identifying a detected level of patient respiratory effort based upon the breath sensor information.

20. The method of claim 19, further comprising, in response to the detected level of patient respiratory effort being outside of a predetermined range, controllably incrementing or decrementing the stimulation intensity of the phrenic nerve stimulation signal by a preset increment value.

21-58. (canceled)
